(19) United States
(12) Patent Application Publication
(10) Pub. No.: US 2025/0339097 A1
(43) Pub. Date: Nov. 6, 2025

(54) NERVE LOCATOR DEVICES

(71) Applicant: Epineuron Technologies Inc., Mississauga (CA)

(72) Inventors: Michael Patrick Willand, Oakville (CA); Ax Varshheshkumar Patel, Mississauga (CA); Ahmed Abdel Halim Attia, Burlington (CA); Sergio David Aguirre, Bolton (CA); Peter Paul Muller, Chicago, IL (US); Aaron Benjamin Eiger, Chicago, IL (US)

(21) Appl. No.: 19/268,618

(22) Filed: Jul. 14, 2025

Related U.S. Application Data
(63) Continuation of application No. PCT/US2025/013890, filed on Jan. 30, 2025.
(60) Provisional application No. 63/627,605, filed on Jan. 31, 2024.

(51) Int. Cl. A61B 5/00

(52) U.S. Cl. CPC A61B 5/4893; A61B 5/7455

(57) ABSTRACT

Systems and devices for providing stimulation to target tissue to locate nerves. Devices may incorporate the use of haptic feedback to provide surgeons optimal communication of device status and/or target tissue excitability. Haptic communication may also work in conjunction with visual indicators to provide dual confirmatory responses to the user or signal other information. Haptic communication is also used to be timed with the application of electrical stimulation to elicit a feeling of contraction in the surgeon's hands as they apply electrical stimulation to a target nerve that results in a muscle contraction.

### CROSS-REFERENCE TO RELATED APPLICATIONS

This application in a continuation application of PCT Application PCT/US2025/013890 filed Jan. 30, 2025, claims the benefit of U.S. Provisional Patent Application 63/627,605, filed Jan. 31, 2024 and titled NERVE LOCATOR DEVICES, the disclosures of both of which are incorporated herein by reference in their entireties and made a part of the present application.

### FIELD

This application relates generally to hand-held devices configured to apply electrical stimulation to tissue and methods of their operation. For example, illustrative embodiments include devices, systems and methods for locating, evaluating and/or testing tissue, more specifically including devices, systems and methods that facilitate the identification and functional testing of peripheral nerves.

### BACKGROUND

During surgical dissection, routine electrical testing of tissue to understand the type or composition of tissue is important. This may give a practitioner information about whether or not the tissue is a nerve or contains nerves and may aid in surgical decision-making. One way to perform such testing includes using electrical stimulation devices and methods. Said devices can output electrical waveforms with sufficient energy to elicit a physiological response in a nerve (e.g., an action potential). Action potentials conducted in nerves that carry motor information typically result in a motor or muscular response by the subject being treated that may be visualized by a surgeon or surgical staff in the operating room. Devices that can output electrical stimulation to elicit these responses are found in a variety of sizes ranging from large mains powered table-top or console like devices or battery powered hand-held devices. Large mains powered devices typically incorporate disposable hand pieces that contain conductive elements or probes that are used to transmit electrical stimulation to tissue. Hand-held devices can be single use and can incorporate the conductive elements within the housing. While both sets of devices can output electrical stimulation, large table-top or console devices are typically connected to visual indicators like LCD monitors to convey stimulation parameter information or physiological response information if responses are recorded with a second set of conductive elements. Hand-held devices are typically not connected to any monitors and include a limited number of indicators due to their small size. These indicators may include visual indicators such as LEDs or small character LCDs to convey limited information to the user. However, during routine use of hand-held stimulators, surgical practitioners rarely divert their gaze to observe said indicators. At times when surgeons or other practitioners divert their gaze and visually focus on a device indicator, the spatial positioning of their hands is often changed making it difficult to both look at a visual indicator on device and spatially position a conductive probe in human anatomy with high acuity. Decoupling these tasks through the use of non-visual indicators may be advantageous to a surgical practitioner. With the advance of smartphones and commoditization of non-visual indicators, such as eccentric rotating mass or linear resonance actuator motors used to drive haptic communication, a more advantageous way of transmitting or otherwise providing information to surgical practitioners during the execution of a procedure can be incorporated into medical devices. Thus, a need exists for nerve location devices with enhanced output characteristics and features.

### SUMMARY

According to some embodiments, a method of operating a device configured to apply electrical stimulation to tissue (e.g., nerve tissue) comprises generating a first haptic output signal configured to be detected by a user holding the device, the first haptic output signal being associated with a first condition pertaining to the device or to the electrical stimulation, in response to detecting the first condition, and generating a second haptic output signal configured to be detected by a user holding the device, the second haptic output signal being associated with a second condition pertaining to the device or to the electrical stimulation, in response to detecting the second condition, wherein the first condition is different than the second condition, and wherein the first haptic output signal is different than the second haptic signal.

According to some embodiments, the first haptic output signal is different than the second haptic signal in at least one of: a number of perceived vibrations, a vibration sharpness, a vibration duration, a vibration intensity, and a vibration frequency, wherein the first and second output signals are generated by a single haptic signal generator, and wherein the first condition or the second condition comprises at least one of the following: a device status condition, an alarm condition, an electrical current flow; a change to any one of the amplitude, frequency or duration of the electrical stimulation; a battery depletion; a battery error; an electrode error; an electrode shorting; and a system status change to any one of power status, standby status, wake status and wake time.

According to some embodiments, the first haptic output signal is different than the second haptic signal in at least one of: a number of perceived vibrations, a vibration sharpness, a vibration duration, a vibration intensity, and a vibration frequency.

According to some embodiments, the first and second output signals are generated by a single haptic signal generator. In other arrangements, the first output signal is generated by a different haptic signal generator than the second output signal.

According to some embodiments, at least one of the first output signal and the second output signal is generated using at least one of the following: a linear resonant actuator, a piezo vibration actuator, a linear magnetic ram, and an eccentric rotating mass.

According to some embodiments, the first condition comprises a device status condition. In some embodiments, the first condition comprises an alarm condition. In some arrangements, the first condition or the second condition comprises at least one of the following: a device status condition, an alarm condition, an electrical current flow; a change to any one of the amplitude, frequency or duration of the electrical stimulation; a battery depletion; a battery error; an electrode error; an electrode shorting; and a system status change to any one of power status, standby status, wake status and wake time.

According to some embodiments, the method further comprises dampening vibrations in electrodes of the device at a vibration frequency associated with at least one of the first and second haptic output signals.

According to some embodiments, a nerve location device comprises a body portion configured to be grasped by a user, an electrode assembly (e.g., bipolar electrode assembly) configured to apply electrical stimulation to tissue, and at least one haptic output generator configured to generate a first haptic output signal and at least a second haptic output signal, wherein the first and the at least second haptic output signals are configured to be detected by a user holding the device, wherein the first haptic output signal is associated with a first condition pertaining to the device or to the electrical stimulation, in response to detecting the first condition, wherein the second haptic output signal is associated with a second condition pertaining to the device or to the electrical stimulation, in response to detecting the second condition, wherein the first condition is different than the second condition, and wherein the first haptic output signal is different than the second haptic signal.

In an illustrative embodiment of the present disclosure, a method of operating a hand-held device configured to apply electrical stimulation to tissue, includes: generating with the hand-held device, a first selection of visual and haptic signals associated with a first condition pertaining to the device or to the stimulation, in response to detecting the first condition; and generating with the hand-held device, a second selection of visual and haptic signals associated with a second condition pertaining to the device or to the stimulation, in response to detecting the second condition; wherein the first condition is different than the second condition, and the first selection of visual and haptic signals is different than the second selection of visual and haptic signals.

In another illustrative embodiment of the present disclosure, a hand-held device configured to apply electrical stimulation to tissue, includes: an electrode system configured to apply the electrical stimulation to the tissue; a visual signal generator configured to generate one or more visual signals visibly perceptible by a user of the hand-held device; a haptic signal generator configured to generate one or more haptic signals tangibly perceptible by the user of the hand-held device; and a controller configured to: control the visual and haptic signal generators to generate a first selection of visual and haptic signals associated with a first condition pertaining to the device or to the stimulation, in response to detecting the first condition; and control the visual and haptic signal generators to generate a second selection of visual and haptic signals associated with a second condition pertaining to the device or to the stimulation, in response to detecting the second condition; wherein the first condition is different than the second condition, and the first selection of visual and haptic signals is different than the second selection of visual and haptic signals.

In another illustrative embodiment of the present disclosure, a hand-held device configured to apply electrical stimulation to tissue, includes: means for generating a first selection of visual and haptic signals associated with a first condition pertaining to the device or to the stimulation, in response to detecting the first condition; and means for generating a second selection of visual and haptic signals associated with a second condition pertaining to the device or to the stimulation, in response to detecting the second condition; wherein the first condition is different than the second condition, and the first selection of visual and haptic signals is different than the second selection of visual and haptic signals.

In another illustrative embodiment of the present disclosure, a method of operating a hand-held device configured to apply electrical stimulation to tissue, includes: detecting a trigger condition associated with a predefined tetanic burst stimulation; and automatically applying an electrical tetanic burst signal associated with the predefined tetanic burst stimulation to an electrode system of the hand-held device, in response to the detecting of the trigger condition.

In another illustrative embodiment of the present disclosure, a hand-held device configured to apply electrical stimulation to tissue, includes: an electrode system configured to apply the electrical stimulation to the tissue; and a controller configured to: detect a trigger condition associated with a predefined tetanic burst stimulation; and automatically apply an electrical tetanic burst signal associated with the predefined tetanic burst stimulation to the electrode system of the hand-held device, in response to the detecting of the trigger condition.

In another illustrative embodiment of the present disclosure, a hand-held device configured to apply electrical stimulation to tissue, includes: means for detecting a trigger condition associated with a predefined tetanic burst stimulation; and means for automatically applying an electrical tetanic burst signal associated with the predefined tetanic burst stimulation to an electrode system of the hand-held device, in response to the detection of the trigger condition.

### BRIEF DESCRIPTION OF THE DRAWINGS

These and other features, aspects and advantages of illustrative embodiments of the present application are described with reference to drawings of such embodiments, which are intended to illustrate, but not to limit, the concepts disclosed herein. The attached drawings are provided for the purpose of illustrating concepts of at least some of the embodiments disclosed herein and may not be to scale.

FIG. 1A illustrates a perspective view of a nerve locator device according to an illustrative embodiment, including block diagram depictions of an internal controller and haptic signal generator.

FIG. 1B illustrates a perspective view of a nerve locator with multiple visual indicators.

FIG. 1C illustrates a perspective view of a nerve locator with a visual indicator that surrounds the user input.

FIG. 1D is a block diagram of the nerve locator device of FIG. 1A.

FIG. 2A illustrates a top view of a section of a nerve locator that includes a scalloped grip.

FIG. 2B illustrates an axial view of a section of a nerve locator that includes a scalloped grip.

FIG. 3A illustrates a perspective view of a gripping position for a nerve locator device.

FIG. 3B illustrates a perspective view of an alternate gripping position for a nerve locator device.

FIG. 4 illustrates a profile view of a nerve locator that includes a textured grip located within a scalloped gripping area.

FIG. 5 illustrates a perspective view of a nerve probe with several bends to engage a PCB that is positioned off center from the enclosure.

FIG. 6 illustrates a perspective view of a monopolar nerve locator with a return electrode.

FIG. 7 illustrates a top view of a nerve locator with a high contrast mechanical display indicating a stimulus parameter.

FIG. 8A illustrates a waveform for eliciting a burst type stimulation with haptic feedback.

FIG. 8B illustrates a waveform for eliciting a burst type stimulation with haptic feedback and a detection waveform.

FIG. 9 is a flow diagram illustrating the process of locating a nerve.

FIG. 10A is a flow chart of a tetanic pulse routine executed by a controller of the nerve locator device according to an illustrative embodiment.

FIG. 10B is a flow chart of a condition detection routine executed by a controller of the nerve locator device according to an illustrative embodiment.

FIG. 11 is a flow chart of a first routine executed by the controller of the nerve locator device according to another embodiment.

FIG. 12 is a flow chart of a second routine executed by the controller of the nerve locator device according to another embodiment.

### DETAILED DESCRIPTION

The devices, systems and associated methods described herein may be used during surgical procedures to, for example and without restriction or limitation, locate nerve tissue and/or test nerve tissue excitability. The embodiments disclosed herein can be used for or in connection with peripheral nerves; however, other types of nerves can also be targeted, such as, for example, nerves in the autonomic system or nerves in the central nervous system, as desired or required. For example, peripheral nerves may include the median nerve in the upper limb, the sciatic nerve in the lower limb, smaller nerves (e.g., the intercostal branches in the thorax) and/or any other peripheral nerve. Autonomic nerves may include, by way of example and without limitation, the vagus nerve, postganglionic parasympathetic splanchnic (visceral) nerves, etc. Nerves in the central nervous system may reside or otherwise be located, at least in part, in and/or near the spinal cord or brain.

Accordingly, nerve surgery procedures may utilize a nerve locator device or system that outputs electrical stimulation to a one or more electrodes (e.g., on a probe) in order to test tissue (e.g., to determine if it is a nerve and its excitability if the nerve contains motor axons that are connected to a muscle).

Several embodiments disclosed in the present application are particularly advantageous because they include one, more or all of the following benefits: rapid nerve integrity assessment through the use of a user input located at the distal end of the device that allows one step manipulation of a stimulus parameter; haptic feedback allowing users to perform a nerve location procedure without changing their line of sight while using the device; a combination of haptic and visual feedback providing redundant indicators for the user and nearby personnel; repeated tetanic burst output allowing a user to maintain an electrode in contact with a target nerve and observe functional muscle responses; and symmetrical housing design that may facilitate different grip orientations without requiring two hands.

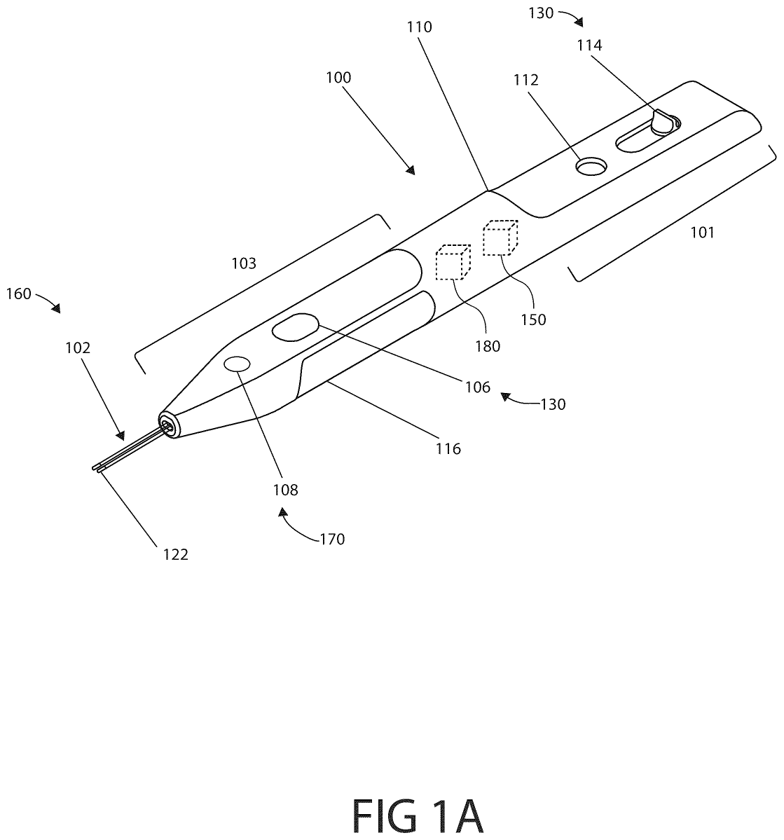

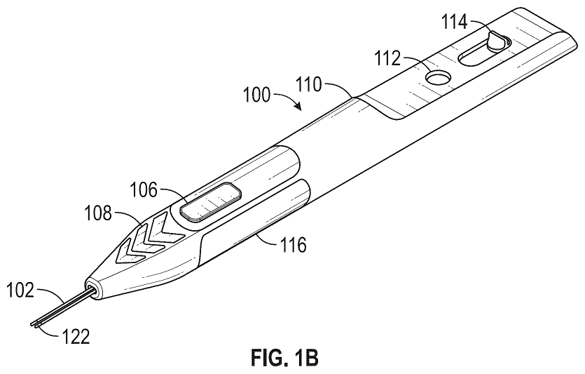

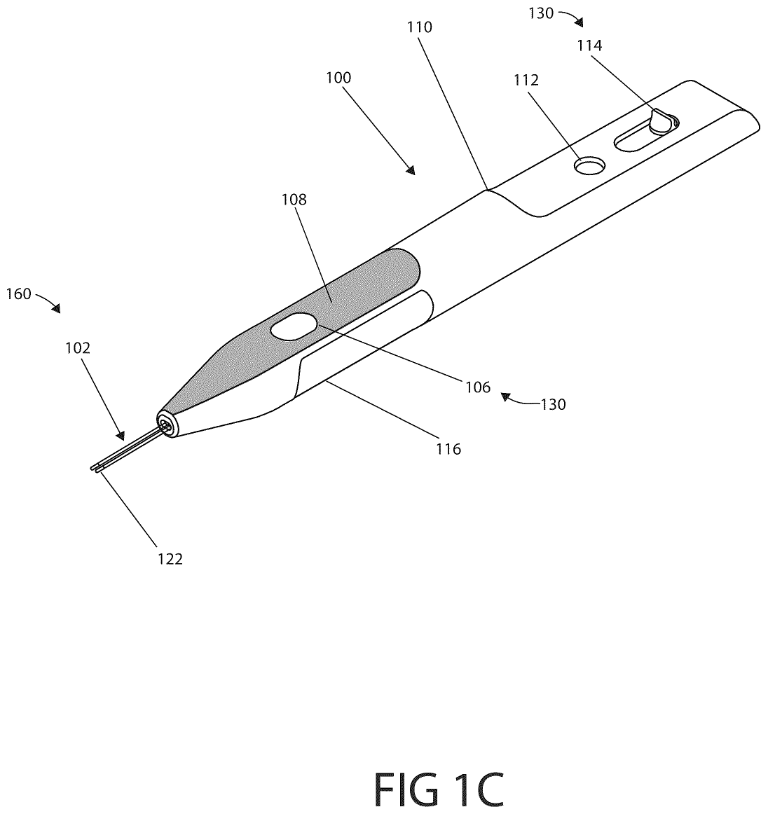

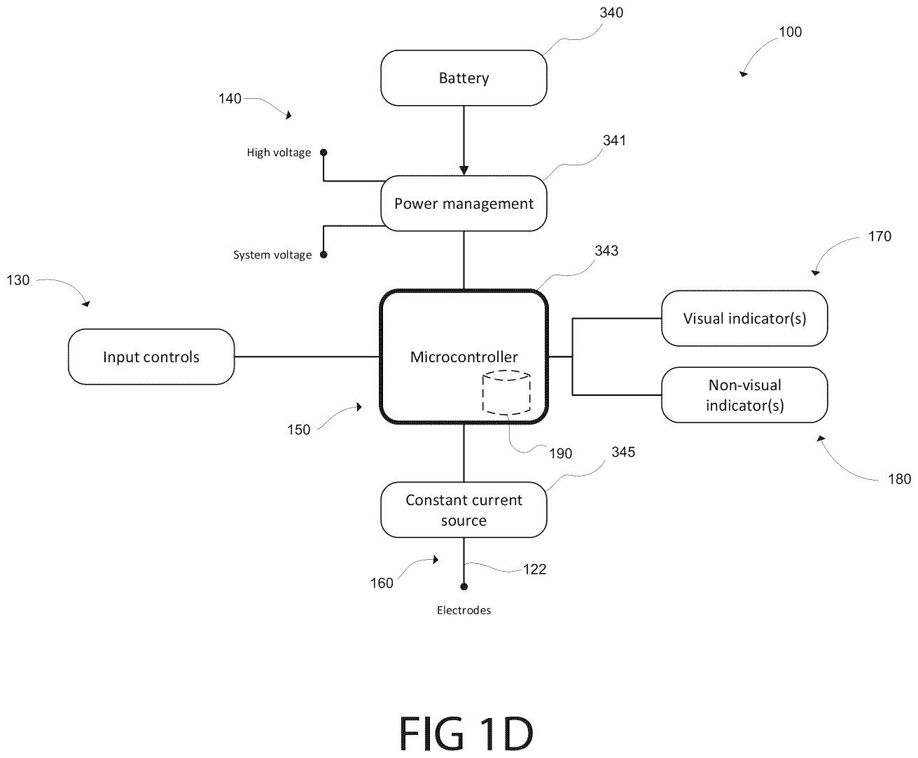

Referring to FIG. 1A, a device (e.g., a hand-held device) configured to apply electrical stimulation to tissue according to a first illustrative embodiment of the present disclosure, is shown generally at 100. In this embodiment, the device 100 includes an electrode system 160 configured to apply the electrical stimulation to tissue, a visual signal generator shown generally at 170 configured to generate one or more visual signals (e.g., signals that are visibly perceptible by a user of the hand-held device), and a haptic signal generator 180 configured to generate one or more haptic signals (e.g., signals tangibly perceptible by the user of the hand-held device).

However, in some embodiments, the device (e.g., hand-held device) 100 comprises a haptic signal generator 180, but not a visual signal generator. Such a configuration can apply to any of the embodiments disclosed herein or equivalents thereof. Accordingly, in some embodiments, the device 100 includes an electrode system 160 configured to apply the electrical stimulation to tissue, and a haptic signal generator 180 configured to generate one or more haptic signals (e.g., signals tangibly perceptible by the user of the hand-held device).

In some embodiments, the device 100 further includes a controller 150 that is programmed or otherwise configured to: control the visual and/or haptic signal generators 170 and 180 to generate a first selection of visual and/or haptic signals associated with a first condition pertaining to the device or to the stimulation, in response to detecting the first condition; and control the visual and/or haptic signal generators 170 and 180 to generate a second selection of visual and haptic signals associated with a second condition pertaining to the device or to the stimulation, in response to detecting the second condition. In some embodiments, the first condition is different than the second condition, and the first selection of visual and/or haptic signals is different than the second selection of visual and/or haptic signals.

Also in this embodiment, the controller 150 can be configured to: detect a trigger condition associated with a predefined tetanic burst stimulation; and apply (e.g., automatically apply) a signal (e.g., an electrical tetanic burst signal) associated with the predefined tetanic burst stimulation to the electrode system 160 of the hand-held device 100, in response to the detecting of the trigger condition. Components and functions of illustrative embodiments of the device 100 and of the controller 150 are described in greater detail below.

### Housing

In some embodiments, the nerve locator device 100 includes one or more of the following: a housing, input controls 130, an indicator or other output (e.g., visual indicator 108, a haptic output or another non-visual output, etc.), a microcontroller, a processor, stimulation output circuitry, a probe, an electrode and/or the like. For example, as illustrated in FIG. 1A, in some embodiments, the locator or locator device 100 comprises the described components and is shown with two nerve probes 102 that include electrodes 122.

In some embodiments, the nerve locator device 100 includes one or more gripping sections 116 that have scallops, recesses and/or similar gripping features 126. Such a configuration can be advantageous as it allows a surgeon or other user to, for instance and without limitation, grip, grasp and/or hold the device in multiple orientations. For example, some surgeons may hold the device using or in a writing grip or pencil grip (e.g., with a finger (e.g., index finger) placed near a first user input selector 106 of the input controls 130). Others may hold the device such that their thumb is positioned on or near the user input selector 106. In some embodiments, the use or inclusion of scallops or similar gripping features 126 reduces the overall diameter or other cross-sectional dimension of the nerve locator device in that section, thereby allowing or facilitating ease of transition between grip orientations. In some embodiments, changing grip occurs during procedures when access to the target tissue is limited or requires repositioning of the surgeon's upper extremity.

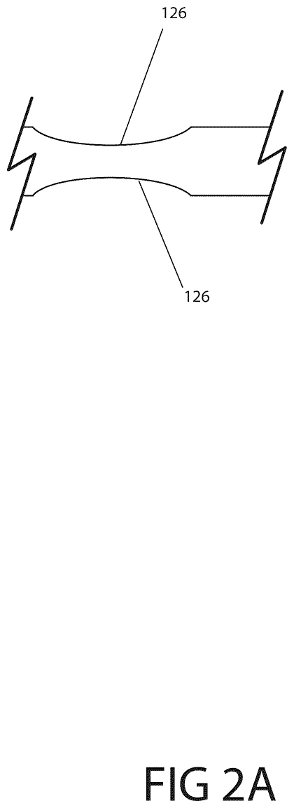

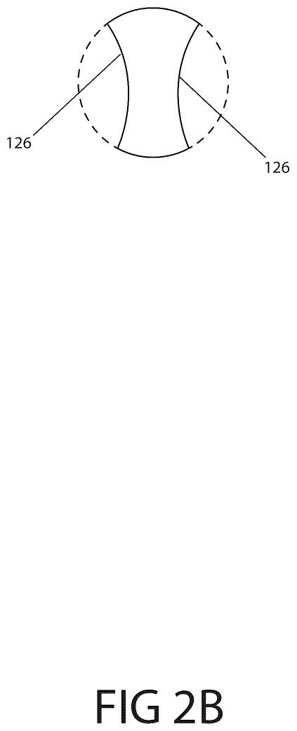

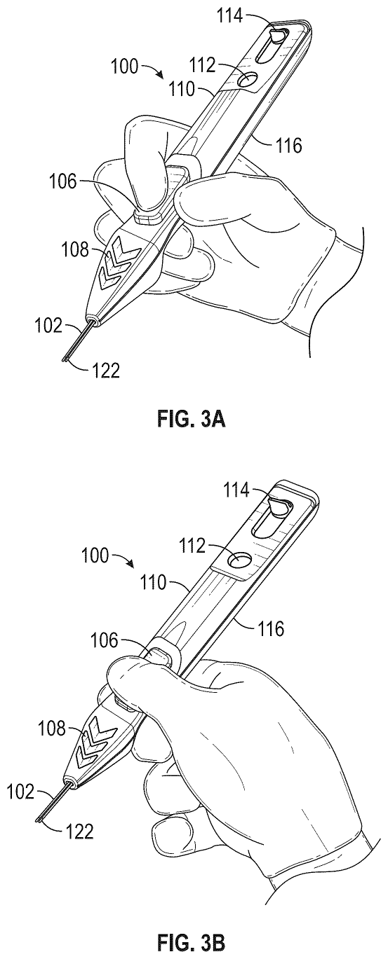

In some embodiments, with continued reference to FIG. 1A, the gripping section 16 may be shaped in multiple planes to create a tri-lobe (or other multi-lobe) form. For example, from a top view, the scallops or similar gripping features 126 may be curved towards the midline of the body. In an axial view, the scallops or similar gripping features 126 may be curved towards the bottom of the device. This is visualized, by way of example and without limitation, in a section view of the locator in FIG. 2A and an axial view in FIG. 2B. This can be advantageous since when a user holds the device in a pencil grip, a curved portion of the housing is resting on the medial aspect of the user's middle or ring finger, and the thumb can comfortably rest on the opposite side curved portion as shown in FIG. 3A and FIG. 3B.

In some embodiments, the proximal end or portion of the nerve locator device may be shaped substantially different than the distal aspect or portion 103 creating a distinct visual hierarchy providing users with a clear understanding of the intended manner of use of the device. In one example, as shown in FIG. 1A, the distal aspect or portion 103 may include a cylindrical or substantially cylindrical shape, and the proximal aspect or portion 101 may include a flattened or substantially flattened (e.g., non-cylindrical) shape or section. The flattened section can be configured such that it facilitates the communication of information to and/or from the user (e.g., through a visual indicator that may be mechanical or electrical such as an LED, using one or more user controls, etc.). This can be advantageous as the flattened section allows for a discrete grip of the device to interact with a user control, and allows for a greater surface area to communicate information versus a cylindrical shape and/or the like.

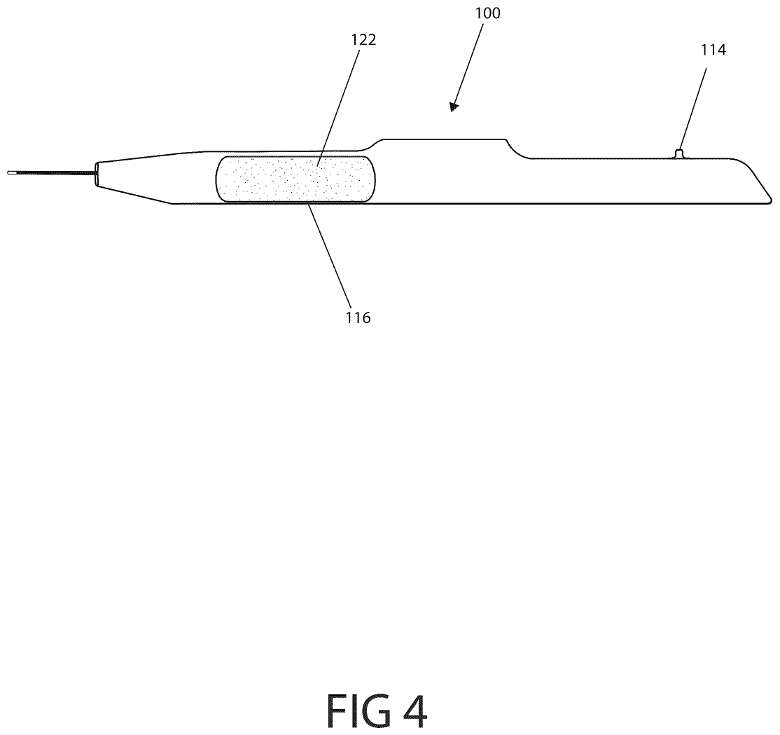

In some embodiments, the scalloped area or portion of the gripping section 116 may include at least one covering or layer having a different material and/or another different property (e.g., smoothness, surface features, etc.) than the underlying surface of the device (e.g., to aid in gripping, handling, etc.). Such material can include an elastomer that is over molded. In some arrangements, the gripping section may include an injection molded texture, as shown in, for example and without limitation, FIG. 4.

In some embodiments, the device includes a transition (e.g., a transition zone 110) between the proximal and distal portions. Such a transition can include a ramp up or ramp down in the shape. In some embodiments, this transition zone 110 can help address one or more purposes and/or provide one or more advantages and benefits. For instance, the transition zone 110 can help direct a user's attention to where to hold the device thus increasing usability and ergonomics of the device. Thus, in some embodiments, the device is configured (e.g., via its exterior shape, curves, recesses, protruding portions, transitions or other features, etc.) to encourage surgeons or other users to grasp the device along a particular area or portion (e.g., the scalloped or gripping portion of the device).

In some embodiments, the bottom or underside of the housing is flat or substantially flat. In the arrangement illustrated in FIG. 1A, for example, the bottom or underside of the housing is the surface opposite the “top” surface, which includes the user input controls 130 (including, for example and without limitation, the user input selector 106 and the switch 114) and the flattened portion. This can be advantageous as it prevents or reduces the likelihood of the device rolling and obscuring any visual information that may be on the top of the device. In some arrangements, the distal aspect or portion 103 of the device is cylindrical or substantially cylindrical with a flattened bottom. In some arrangements can be advantageous as, for example, placement of the device upside down or on its side results in the device rotating to be upright (or another desired orientation).

### Electrode System

In some embodiments, the electrode system 160 of the device 100 includes one or more nerve probes 102 that comprise one or more electrodes 122. In some arrangements, the electrodes 122 have or are positioned in a bipolar configuration as shown, by way of example and without limitation, in FIG. 1A. Said electrodes may include a circular or cylindrical, square, triangular, other polygonal, irregular or any other shape. In some embodiments, the electrodes have identical or substantially identical diameters (or other cross-sectional dimensions), lengths, shapes, orientation, material(s), electrical properties and/or the like. However, in other arrangements, one or more properties or aspects of the electrodes (e.g., diameter or other cross-sectional dimension, shape, length, material, electrical properties, etc.) are different between adjacent electrodes, as desired or required. The electrodes may be at least partially insulated (e.g., electrically) to expose varying amounts of the conductive portion of the electrode. In some embodiments, in order to create a bipolar electrical field during use of the nerve locator device, the electrodes may be spaced relatively closely together ranging from 0.1 to 20 mm (e.g., 0.1, 0.2, 0.3, 0.4, 0.5, 1.0, 1.1, 1.5, 5.0, 5.5, 10, 15, 20 mm, 0.1 to 5, 0.1 to 10, 5 to 10, 10 to 20, 5 to 20, 0.1 to 15 mm, values between the foregoing spacings or ranges, etc.).

In some embodiments, the controller 150 may be configured to adjust a current density of the electrical stimulation, e.g., in response to user input. For example, in one such arrangement, the spacing between the bipolar electrodes is adjustable, allowing the user to configure or customize the current density. This can be accomplished by, for example and without limitation, moving the probes closer together or farther apart relative to one another, otherwise modifying the distance between probes, etc.

In some embodiments, spacing between the electrodes may be adjusted manually (e.g., by the physician or other user) by using softer metal or a smaller probe diameter. In other embodiments, spacing may be determined by use of an insert between probes that fixes the spacing to a pre-determined distance. In some embodiments, removal or other manipulation of this insert allows the probes to be moved closer together, whereas replacement of the insert and other manipulation of the insert and/or the probes restores, at least in part, the original separation spacing or distance.

In some embodiments, the electrodes comprise stainless steel (e.g., 304 stainless steel) and/or other conductive materials such as platinum, platinum-iridium, silver, copper, carbon black, other metals and/or alloys, etc.

In some embodiments, the electrode(s) is/are insulated using an insulative material such as FEP, PTFE, PVDF, HDPE, etc. In some arrangements, the insulative material is applied via heat shrinking methods, dip coating, extrusion coating, any other application technology and/or a combination thereof.

In some embodiments, the nerve probe 102 is straight (e.g., linear) or substantially straight or linear. However, in other arrangements, the nerve probe 102 is non-linear or non-straight. For example, the probe 102 can be bent at a desired angle relative to horizontal (e.g., the longitudinal axis of the device), such as, for example and without limitation, 0 to +−90° (e.g., 0.1, 0.2, 0.5, 1.0, 1.5, 2.0, 5.0, 10, 15, 20, 25, 30, 50, 55, 60, 65, 75, 80, 90°, 0 to 10, 10 to 20, 20 to 30, 0 to 30, 30 to 60, 60 to 90, 30 to 90, 0 to 45, 45 to 90°, angles between the foregoing values or ranges, etc.), can be curved or non-linear (e.g., with a constant or varying radius of curvature).

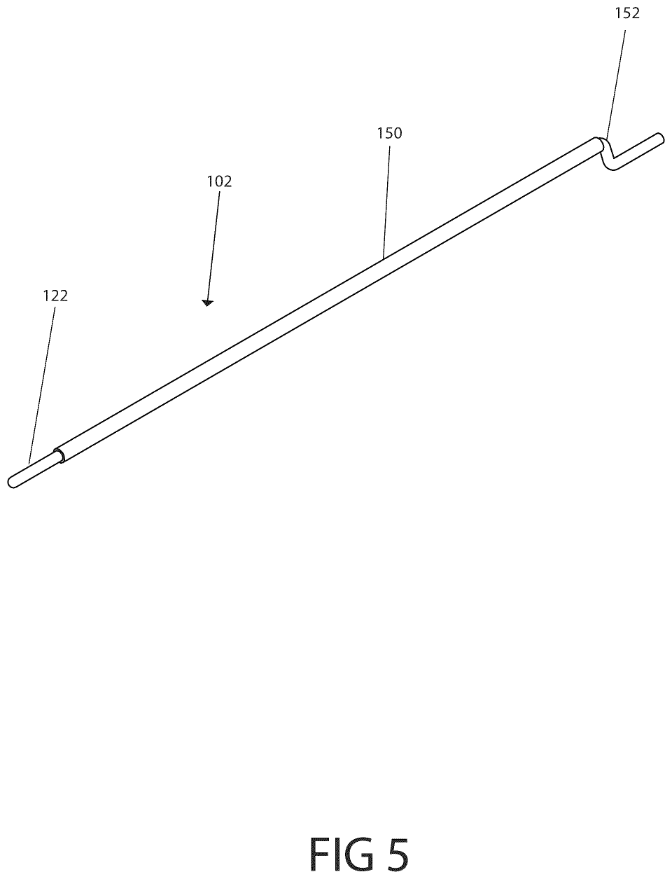

In some embodiments, the nerve probe 102 is required to be bent to interface with the printed circuit board within the housing. Depending on the placement of the PCB within the enclosure, a straight line path from PCB to probe exit on the enclosure may not exist and requires the probe to be bent within the enclosure in order to engage the appropriate probe connector located on the PCB. FIG. 5 shows an example of a probe 102 that has several bends 152 in order to engage a PCB that is not located at the midline of an enclosure. In some embodiments, placing the PCB away from the midline may allow more room within the enclosure for battery placement, motor placement, button placement, or the like.

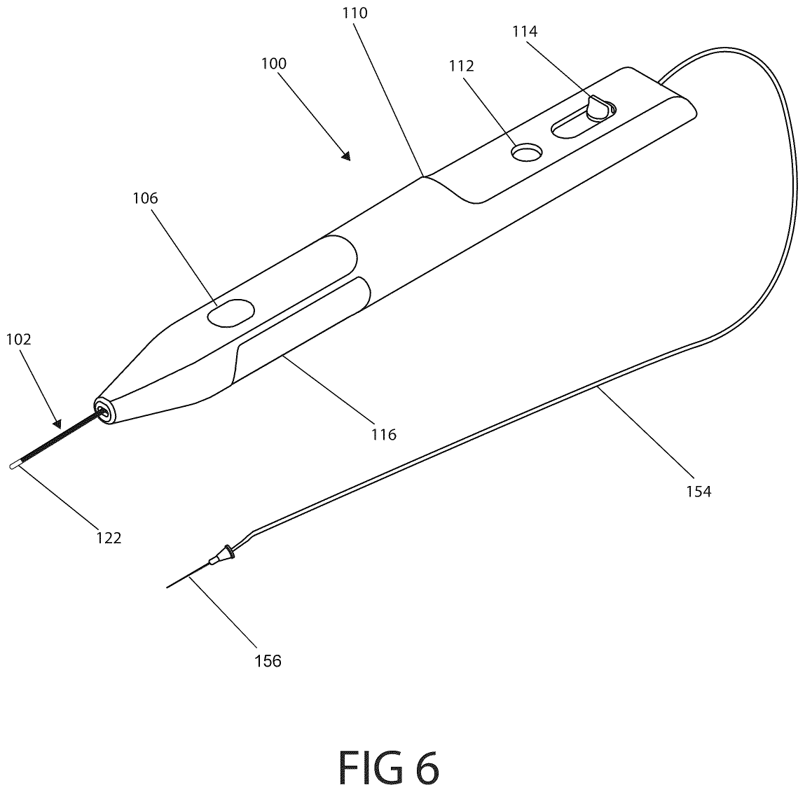

In some embodiments, only one or more electrodes are used, together or individually, in a monopolar electrode configuration. In such arrangements, the electrode(s) are configured to be used together with a return electrode. In some embodiments, such a return electrode is either connected directly to the circuit board within the housing of nerve locator or via a connector that communicates externally. FIG. 6 shows an example of a PCB connected return electrode with a single monopolar electrode configuration.

In some embodiments, a connector may allow for connection of not only a return electrode but other types of electrodes, such as, for example and without limitation, cuff electrodes, needle electrodes, other subcutaneously positioned electrodes, surface electrodes and/or other type of electrodes.

In some embodiments, said electrodes connected to the connector may have multiple contacts, allowing the device to be used in a bipolar, tripolar, or other electrode configuration, as desired or required.

### Controls

In some embodiments, the nerve locator device 100 includes one or more user input controls or selectors 130, such as, for example and without limitation, a switch and/or other component 114 for adjusting one or more stimulation and/or other operational parameters of the nerve locator device. These parameters can include, for example and without limitation, stimulation amplitude, pulse width or duration, output energy, frequency, burst frequency or the like. In some embodiments, the device includes two or more controls. In such a configuration, each control can be configured to regulate a different operational parameter of the device and/or two or more aspects of an operational parameter but in a different way.

In some embodiments, a control or selector include a force or touch sensor, a capacitive or resistive element connected to a circuit (e.g., to determine changes in capacitance or resistance), a scroll wheel, a potentiometer, and/or the like.

In some embodiments, the controls for stimulation parameters may include or resemble a sliding multi-pole switch 114 as shown, for example, in FIG. 1A.

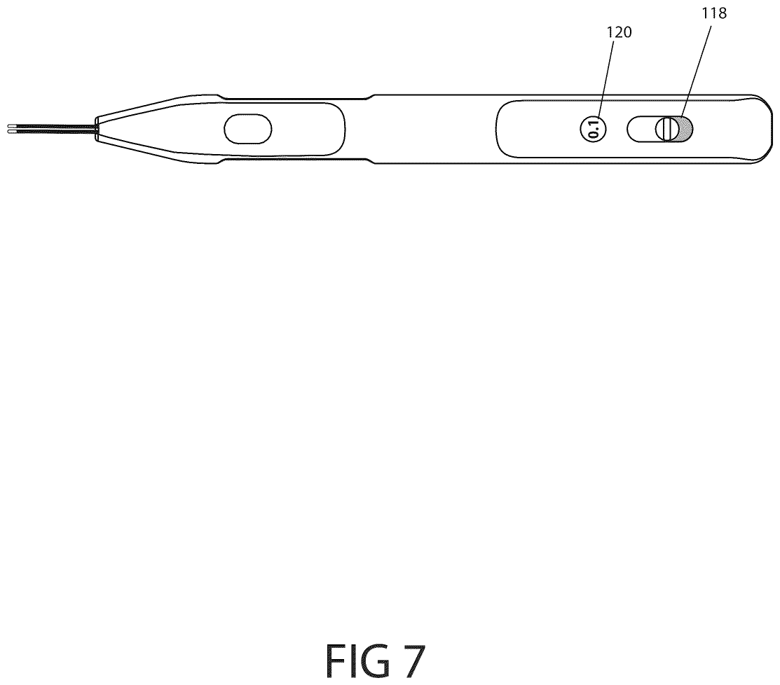

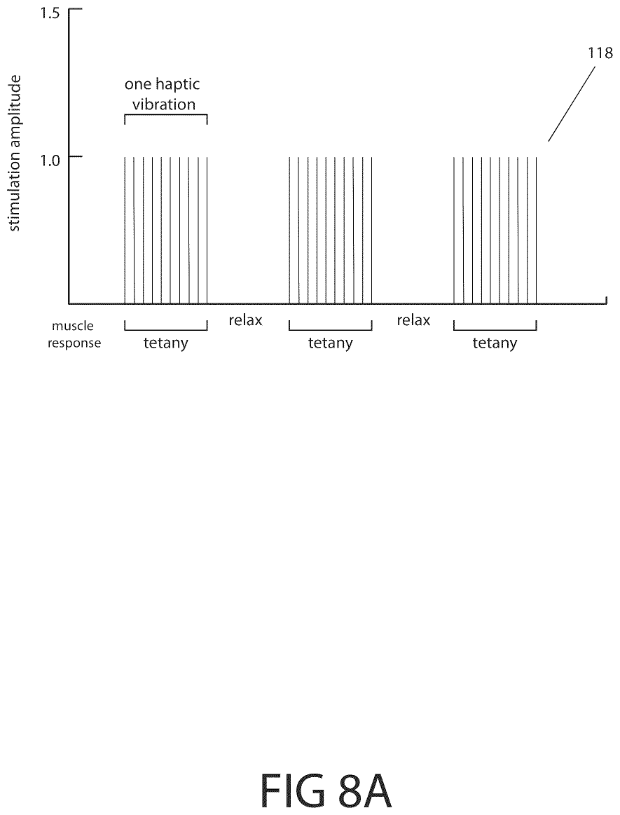

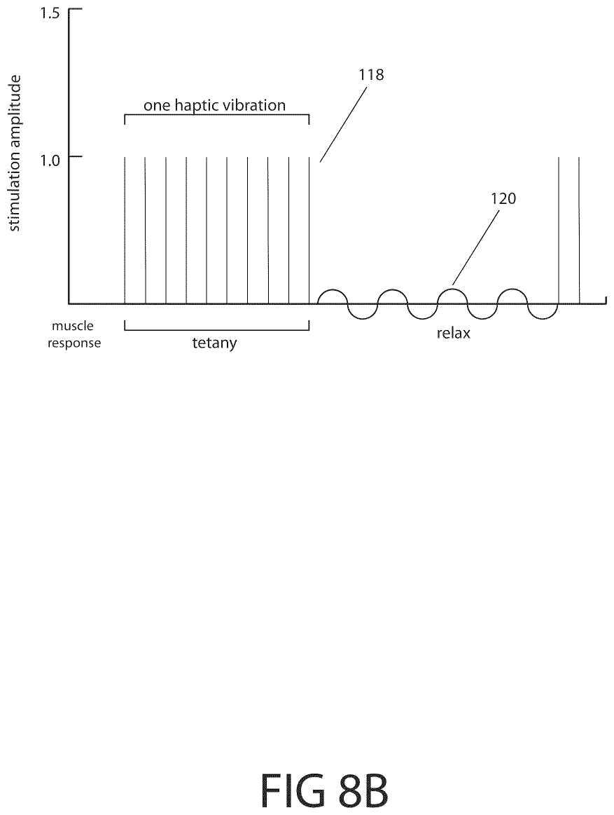

With continued reference to FIG. 1A, the switch 114 may be configured to connect or otherwise operatively couple to a display or other output to indicate, by way of example, the parameter that is currently engaged. Such a display or output can be included on the device. Alternatively, the display or other output can be included as part of a separate device or system (e.g., a monitor, an output of a separate device, such as a smartphone, tablet, other computing device, etc.). The switch 114 may also be coupled (e.g., mechanically) to a sliding mechanism to display the current parameter engaged in a display window 112 as shown in FIG. 7. This mechanism can allow for optimal, improved or enhanced contrast in the operating theatre as high powered surgical lights may make viewing of LCDs or other electronic displays difficult.

With continued reference to stimulation controls, in some embodiments, the nerve locator device 100 may include one or more user input controls 130, such as the user input selector 106. Such a user control can be located on or near the distal aspect or portion 103 of the housing. This switch or other control can include a momentary push switch that is used to change (e.g., rapidly change, for example, relative to a slower rate of change enabled for the device) one or more operational parameters of the device. In one embodiment, the location of this switch or other control is advantageously placed where a user would be able to use the device with only one hand. In one example, the user can manipulate the control (e.g., button, dial, switch, etc.) using only their index finger or thumb, while holding the device with the same hand. In some arrangements, the switch or other control includes a capacitive or resistive type switch or the like.

In a non-limiting example, the user input selector 106 is a switch or other manipulatable controller and is used to change the stimulation amplitude by a predetermined amount, for example, doubling or tripling an amount (e.g., a baseline amount). However, in other arrangements, the amount of change can vary (e.g., along a continuous spectrum or non-discrete levels). For example, the device can be configured to permit a user to select other and/or additional discrete levels of stimulation amplitude. This may be advantageous to a user that is probing tissue but is uncertain of the stimulation induced response. In such a case, the user may want to increase (e.g., relatively quickly increase, for example, relative to a slower rate of change enabled for the device) the stimulation output to ensure the response is more visible or to confirm that there is no response. Such ‘one touch’ or ‘single touch’ stimulus ‘boosting’ can reduce time spent by a user where switching parameters on traditional devices may require a second hand or a second user.

In some embodiments, the user input selector 106 may require a predetermined amount of force or pressure in order to actuate or otherwise activate. Lower force levels may be advantageous to prevent or reduce the likelihood of transmission of push button force towards the distal probe. For example, when probing tissue under a microscope and when it is desirable to boost the stimulus amplitude, the user may press the switch or otherwise activate the controller. In some embodiments, with a sufficiently low actuation force, the distal aspect of the probe may not move or may move only minimally, whereas a high actuation force switch will have the push button force displace the probe potentially from the field of view, may damage tissue and/or result in other undesirable consequences. Desirable forces may range from 20 g to 300 g (e.g., 20, 20.1, 20.2, 20.5, 21, 22, 23, 25, 30, 35, 40, 50, 60, 70, 100, 150, 200 g, 20 to 200, 20 to 50, 50 to 100, 20 to 100, 100 to 300 g, forces between the foregoing values or ranges, etc.).

### Visual Output/Signal Generator

In some embodiments, a nerve locator device 100 comprises at least one visual signal generator 170. The visual signal generator 170 can include one or more visual indicators 108 and/or other visual outputs configured to communicate information to the user. The visual indicators or outputs 108 may include one or more light-emitting diodes (LEDs) for example, and are used to communicate information related to a procedure involving the device 100 (e.g., regarding the delivery of stimulation to a target nerve). In one example, LEDs (e.g., placed on a printed circuit board) may be one color or may have multiple (e.g., two or more) colors (e.g., RGB type) within one part. The output of the LEDs may pulse or flash at the rate of stimulus delivery or another frequency or in some examples may be constant (e.g., non-pulsing, non-flashing, etc.), as desired or required. The arrangement of visual indicator 108 LEDs in one embodiment, as illustrated in FIG. 1B, may be collinear or substantially collinear such that they may produce a chasing effect (e.g., a first LED is illuminated, followed by the next one, followed by the next one, etc.). The LEDs may also interface light pipes or similar features to help funnel or otherwise direct light to the exterior surface of the housing. Such piping or other features can help maintain desired or required (e.g., relatively high) brightness levels (e.g., a level at or above a threshold brightness level) in the face of very bright surgical lights in the environment in which the nerve locator device may be used. In some embodiments, the LEDs are configured to be visible to a user even in environments that otherwise may overpower or perceptually dim the visual output of the nerve locator device. In some examples, light piping is not used (or the lighting effect is attenuated) and/or the housing wall thickness may be reduced to create an effect where the housing itself is illuminated.

In some embodiments, the distal aspect 103 of the nerve locator housing may be illuminated completely or substantially completely. This may be advantageous as the surface area of emitted light is much larger than that provided by discrete light pipes. The larger emitting surface may be more visible at lower brightness levels than a much more focused source with higher brightness arising from a light pipe. In some arrangements, as surgeons or other users of the nerve locator device typically focus on the distal electrode probe, their visual field is limited and any visual cues from the device would be noticed only in their peripheral vision. A larger source may be able to capture a surgeon's peripheral vision better than a smaller light source.

In one example, the visual indicator may also indicate when the momentary switch and/or other control of the device has been pressed. This may be accomplished through a change in color, a change in output frequency, a change in brightness, a change in output pattern and/or a combination of these elements, as desired or required. A change in one or more of such parameters may achieve different visual effects, such as, for example and without limitation, a breathing effect, glowing effect, dimming effect, pulsing effect, chasing effect, etc., as desired or required. In some embodiments, the visual effects can be customized, modified and/or otherwise adjusted (e.g., by the manufacturer, the user, etc.).

In some embodiments, the arrangement of visual indicators 108 is such that the indicators 108 are positioned or biased, at least partially, around one or more input controls 130, such as, for example, a user input selector 106. This arrangement may result in an effect that surrounds, at least partially, the user input, as shown in FIG. 1C, for example. With further reference to the embodiment illustrated in FIG. 1C, the visual indicator 108 is depicted by the shaded area which is the area of visual illumination. This area of visual illumination may be enabled by using one or more LEDs and/or other light sources that are placed at, adjacent and/or near (e.g., directly underneath) this area in order to maximize or otherwise enhance light transmission. In arrangements where more than one LED or other light source is used, the LEDs and/or other light sources may be turned on/off or otherwise activated/deactivated in a sequence (e.g., to resemble a chasing effect as previously described, to create another desired effect, etc.). In some arrangements, such an effect (e.g., a chasing effect, other “moving” effect, etc.) may be synchronized with the delivery of electrical stimulation, and in arrangements where LEDs are placed not only around the user input selector 106 but also towards the probe, a chasing effect can provide the appearance of something being emitted from the device (e.g., light starts at the user input and progresses towards the distal end of the locator device).

In some embodiments, the visual indicator is located or placed at, adjacent or near the electrode so that it is closer to the surgeon's field of view. This may be accomplished, for example and without limitation, by piping or otherwise directing light from a circuit board through one or more light piping elements and/or other features towards the electrode. In one example, the nerve probe passes at least partially through a channel in a cylindrical light pipe.

### Haptic Output/Signal Generator

In some embodiments, in addition to or in lieu of visual output/indicators, the nerve locator device 100 includes one or more haptic outputs (e.g., a signal generator 180), which comprise(s) one or more haptic communication devices, components and/or features. Such haptic devices may include a vibratory motor, a linear resonant actuator, an eccentric rotating mass, a linear magnetic ram, a piezo vibration actuator and/or any other device or component configured to generate a vibratory or haptic effect. Such haptic devices and/or components, or equivalents thereof, may be used in conjunction with visual indicators 108 to communicate a status of the device, a change in stimulus parameters, and/or other occurrence, event and/or variable. In other embodiments, haptic indicators may function in lieu of visual indicators rather than supplementing them. As noted herein, in yet other embodiments, one or more visual outputs are used alone, without the use of haptic output. For any of the embodiments disclosed herein or equivalents thereof, a nerve locator device can include another type of output (e.g., audible) with haptic and/or visual outputs, as desired or required.

In some embodiments, one or more haptic motors may be mounted at least partially inside the housing, and in some arrangements, the mounting position may be dictated or determined, at least in part, by the hand position of the user. For example, in some embodiments, when the mid-body of the nerve locator device 100 is in contact with the first dorsal interosseous muscle, positioning a haptic motor in, along or near the mid-body of the locator would transmit vibratory signals to the user in a desired manner (e.g., with a desired ore required effect).

In some embodiments, mounting or other positioning of the motor takes into consideration the possibility that vibrations created by the motor may be undesirably transmitted to certain areas of the device, such as, for example, the nerve probe. Haptic communication or impact that interferes with the nerve probe 102 may become problematic during procedures where a relatively high level of steadiness and precision are required or desirable (e.g., when the locator is used under a microscope or with loupes, where a greater level of precision at the target nerve site is necessary or beneficial, etc.). Accordingly, for any of the embodiments disclosed herein and equivalents thereof, a nerve locator device can be configured to at least partially dampen, reduce or remove vibration and other movements (e.g., originating from a haptic motor, other source of mechanical energy, etc.). Mechanical dampers and/or other dampening components, devices and/or features are designed to reduce the transfer of vibration between interconnected components by absorbing at least a portion of the kinetic energy that is generated by the vibrating component.

Such damping can be accomplished by incorporating one or more damping elements or technologies within the nerve location device. In some embodiments, at least one mechanical damping element may be placed between different device components to prevent or reduce the likelihood of vibrations from being transmitted to one or more electrode(s) and/or other portions of the nerve location device where a reduced impact of vibration or haptic effect are desired or required. For example, damping elements may be strategically located or placed between one or more and/or adjacent one or more of the following locations: the haptic motor and the PCB; the motor and the enclosure; the PCB and the enclosure; the electrodes and the enclosure; the electrodes and PCB; lining the entire inside of the enclosure.

In some embodiments, the mechanical damping elements may comprise an isolation pad, mount, spring, or combination of these components. The mechanical damping elements can comprise one or more viscoelastic materials (e.g., rubber, cork, neoprene, foam, Sorbothane®, silicone, etc.), other relatively soft and/or insulative materials, etc., to reduce (e.g., diminish, abolish, attenuate, etc.) vibration effects. In some embodiments, an adhesive (e.g., glue, tape, potting compound, etc.) may be used to secure the motor or other components of the device in order to minimize or otherwise reduce vibration of components and the transmission of vibration to the electrodes. The use of such adhesives and/or other materials may be placed between the PCB and the enclosure, the motor and the enclosure, the PCB and the motor, at one or more location(s) or point(s) where the electrodes interface with the enclosure or the PCB and/or at any other location, as desired or required.

In some embodiments, mechanical fasteners may be used to secure components of the device, either in addition to or instead of an adhesive. Such mechanical fasteners may comprise screws, standoffs, ribs and/or the like. According to some embodiments, the motor may be secured such that it is in direct contact with the enclosure and avoids contact with the PCB, which may be physically connected to the electrode. However, in other arrangements, the motor is secured such that it is in indirect contact with the enclosure, as desired or required. In some embodiments, the PCB may be secured to the enclosure, using various methods described herein, such that the transmission of vibration and/or other movements from the motor to the probe (e.g., and thus, electrodes positioned along the probe) can be reduced or eliminated.

In some such embodiments, the device 100 may further include a vibration dampener configured to dampen vibrations in the electrode system of the device at a vibration frequency associated with at least one of the selections of visual and haptic signals described herein. In some embodiments, each of such signals can be associated with a respective condition pertaining to the device 100 or to the stimulation. For example, in one embodiment, the vibration dampener includes one or more insulative vibration-dampening materials in physical (e.g., direct or indirect) contact with at least a portion of each electrode 122 of the electrode system 160.

In some embodiments, the geometry of the enclosure or electrode may be designed to minimize or otherwise reduce vibration. In some arrangements, the diameter (or other cross-sectional dimension and length of the electrode may be altered to change the stiffness, cross sectional area and/or other physical properties, which can result in reduced vibration of the electrode. In one example, the electrode may have a larger diameter or other cross-sectional dimension at, towards or near the end that interfaces the PCB, thus increasing stiffness and reducing vibration amplitude.

In some embodiments, the wall thickness of the enclosure near the probe may be altered, ribs or similar members or features may be added to the walls of the enclosure and/or other changes can be implemented to increase the stiffness and reduce vibration amplitude.

In some embodiments, the haptic motor may be driven at a frequency that differs from the resonant frequency of the materials that comprise the enclosure, PCB, and/or electrode. This may minimize or otherwise reduce the amount of vibrational energy absorbed by the enclosure, PCB, and/or electrode from the vibration of the motor. In some arrangements, the motor may be driven at a specific amplitude wherein the vibrations are still felt by the user but are minimized or otherwise reduced at or near the electrode. Such vibration dampening features can be incorporated into any of the device embodiments disclosed herein and equivalents thereof.

In some embodiments, multiple (e.g., two, three, more than three, etc.) haptic motors are used in a single device. In such embodiments, the haptic signal generator 180 may include a first haptic signal generator configured to generate a first haptic signal of a first selection of visual and haptic signals, and may further include a second haptic signal generator different than the first haptic signal generator, the second haptic signal generator configured to generate a second haptic signal of a second selection of visual and haptic signals. Such motors can be identical or similar to each other. However, in other embodiments, at least two different varieties or types of motors are used in a single device (e.g., to convey or otherwise incorporate features that are advantageous to each motor). For example, in some embodiments, the controller 150 is configured to control the haptic signal generator 180 to generate a first selection of visual and/or haptic signals by generating a first haptic signal, which includes a first vibration signal within a first frequency range. In some of such embodiments, the controller is configured to also generate a second selection of visual and/or haptic signals by generating a second haptic signal that includes a second vibration signal within a second frequency range different than the first frequency range. Although different haptic vibration frequencies can be achieved with a single haptic motor, in some embodiments, a plurality (e.g., 2, 3, more than 3, etc.) of haptic motors can be used, including for example a linear resonant actuator to communicate very fine or high-quality vibrations, in combination with an eccentric rotating mass to output higher-power, more crude signals. In some arrangements, the eccentric rotating mass may communicate alarm-type signals to the user (e.g., using a first haptic output), while the linear resonant actuator may communicate device status (e.g., using a second haptic output that is different, in at least one manner (e.g., type, speed or frequency, duration, sharpness or intensity, etc.) relative to the first haptic output) to the user. In other similar embodiments, each of the first haptic signal generator and the second haptic signal generator includes a different selection from the group consisting of: a linear resonant actuator, a linear magnetic ram, a piezo vibration actuator, and an eccentric rotating mass.

In some embodiments, the haptic signal generator 180 is configured to generate a sequence of haptic signals at different locations in the device 100. To achieve this, the haptic signal generator 180 may include two or more motors to communicate (e.g., via haptic output) information related to the flow of current down or through the device. In some arrangements, where each motor is activated sequentially, this concept may be known as vibrotactile flow.

In some embodiments, the parameters that dictate or otherwise control, at least in part, haptic output may include the number of perceived vibrations (e.g., single, double, triple, quadruple, etc.), the sharpness of the vibration (e.g., how fast or slow the vibration starts or stops or reverses polarity), the vibration duration, the vibration intensity, the vibration frequency or frequency of vibrations, any other property associated with vibratory movement, etc. In some embodiments, different predefined selections or combinations of visual and/or haptic signals are associated with different respective conditions pertaining to the device 100 or to the stimulation of the tissue by the device. In such embodiments, therefore, to help differentiate a first selection of visual/or and haptic signals associated with a first condition from a second selection of visual and/or haptic signals associated with a second condition, a first haptic signal of the first selection may differ from a second haptic signal of the second selection in at least one of: a number of perceived vibrations, a vibration sharpness, a vibration duration, a vibration intensity, and a vibration frequency.

In some arrangements, the device is configured to correlate (e.g., directly, indirectly, linearly, non-linearly, etc.) the vibration intensity (and/or another parameter or property related to vibration or haptic output) to the strength of a particular parameter, such as, for example and without limitation, the stimulus output. In some arrangements, the frequency of vibration correlates, at least in part, to the stimulation frequency (e.g., slower vibration for a 2 Hz stimulation output, faster vibration for a 20 Hz stimulation output). In some embodiments, the vibration frequency includes doublet or triplet (or other multiple) vibration patterns (e.g., instead of constant or substantially constant frequency vibration).

In one embodiment, haptic communication may be advantageous during certain procedures where identification of nerve tissue is needed or helpful. For example, haptic communication can be used when locating small motor nerves that require focused attention in the surgical field. In these procedures, a surgeon may need to carefully probe the tissue while trying to observe a muscle response. Often times, a surgical assistant will be observing on the surgeon's behalf and/or as an extra pair of eyes for the surgeon. To solve this issue and to enhance procedures performed using the devices disclosed herein, in some embodiments, providing a non-visual indicator, such as haptic communication, may aid surgeons by allowing them to feel or otherwise sense (e.g., instantaneously or substantially instantaneously, via the haptic output, etc.) when the probe is in contact with tissue and providing adequate stimulation. This can advantageously allow surgeons to strategically divert their attention to the muscle or other tissue of interest while maintaining their hand position. In some arrangements, during the course of the procedure, surgeons are able to obtain (e.g., via haptic feedback and output) information regarding the operation of the nerve locator device (e.g., an appropriate electrical stimulation being delivered).

In some embodiments, haptic communication may be used to provide one or more types of information to the user. In some arrangements, this information includes, by way of example and without limitation, one or more of the following: system status, which may include device power status (e.g., on or off), standby status, wake or wake time, end-of-life or battery depletion, battery error, system voltage error, electrode error, electrode probe shorting, etc.

In one non-limiting example, as the device powers on, at least one vibration or vibration burst is delivered to the user to confirm a proper powering event, to indicate initialization of the system, etc. In another non-limiting example, at least one haptic vibration or vibration burst may be output when the device is turned off, or is in standby, or has transitioned from a different mode such as standby to active or vice versa. In these examples, the haptic waveforms used for communication may be different or the same from each other (e.g., depending on the event (on or off), depending on one or more other events or conditions, etc.), as desired or required.

In some embodiments, vibration sharpness (and/or another vibratory property) may be altered to generate crisper or softer vibrations felt by the end user. In some arrangements, this property is tied or related (e.g., directly or indirectly) to the rise time characteristics of haptic motors. ERMs are DC motors with a counterweight attached to the shaft. In some embodiments, as this weight rotates, unbalanced forces (e.g., centripetal forces) displace the motor (e.g., in two axes) creating a vibration. However, in some embodiments, vibration is produced once the motor reaches its operating speed. By way of example and without limitation, this can take approximately 90 ms or more for brush type ERMs and approximately 70 ms or more for brushless ERMs, which translates into a softer vibration felt by the end user. In some embodiments, double magnet ERMs have considerably better performance at 35 ms or more. In some embodiments, LRA motors operate with a moving mass attached to a voice coil. Driving the coil with an AC signal at its resonant frequency can cause such a motor to vibrate in a single axis. In some embodiments, the rise times for such motors are considerably quicker (e.g., at around 10 ms). In some embodiments, therefore, such motors can provide certain advantages, especially for high quality haptic applications. In some embodiments, ERM and LRA rise and fall times can be improved through overdriving techniques. Initially driving a motor at its maximum operating voltage as opposed to its typical operating voltage can result in faster acceleration of the moving mass and therefore shorter rise times. Reversing the polarity of the voltage can create shorter fall times or quicker braking, in some arrangements. This can be desirable when, for instance, communicating sharp brief vibrations to the end user such as clicks (single, double, triple, etc.).

In some embodiments, haptic communication may be used to provide the user with information related to a change in the device settings. In some arrangements, a change in the device settings may include changes to the stimulation output parameters. In another non-limiting example, a user may incrementally increase the stimulation amplitude output (e.g., using the user input selector 106) from one level to the next incremental level (e.g., 1 mA to 2 mA). The haptic communication or output can include a different pattern and/or other characteristic, such as, for example, a double vibration (e.g., indicated a doubling of the amplitude or a change in setting to 2 mA), another type of vibration pattern and/or the like. In some arrangements, if the user further increases the amplitude to the next level (e.g., 3 mA), the haptic communication or output can change once again, and can, for example and without limitation, include a triple vibration (e.g., three vibrations to indicate 3 mA or a tripling of output). In some embodiments, the controller 150 is configured control the visual and haptic signal generators 170 and 180 to generate or otherwise help create a predefined selection of visual and haptic signals by generating a visual signal using the hand-held device, and synchronizing the visual signal with at least one haptic signal. These haptic outputs may be at least partially synchronized or substantially synchronized with one or more visual outputs (e.g., visual LED outputs) to provide multiple (e.g., double) confirmatory signals about device settings and/or parameters associated with using the device. In some embodiments, such as that shown in FIG. 1B, the visual signal generator 170 is configured to generate a sequence of visual signals at different locations on the device. In some arrangements, haptic outputs may include vibrations specific to increments or decrements of stimulus output. In some embodiments, the vibration amplitude, strength, frequency, duration and/or other property may be correlated to the stimulation output or device settings, either in addition or in lieu of correlation to another parameter or setting. For example, stronger vibrations can be output when the stimulation current is higher or stronger. This can be used in addition to or in lieu of any embodiments disclosed herein (e.g., the above embodiment related to the number of vibrations). Therefore, in some embodiments, the type or pattern, quantity, strength, frequency and/or other haptic output characteristics can be varied, as desired or required, to provide different sensory feedback to a user.

In some embodiments, a relatively strong (e.g., maximum level, higher level setting, etc.) vibratory output may be used to indicate an error and/or other malfunction (or a likely/expected or predicted error and/or malfunction) to the device and/or a procedure in connection with which the device is used. Such high output settings can be reserved for specific situations such as stimulation of muscle directly.

In some embodiments, haptic communication may be used to provide information to the user on a change in an environmental and/or operational condition of the device. For example, in some arrangements, tissue stimulating devices require a conductive medium to pass current through and the state of this conductive medium may be communicated to the user through haptic output. In one non-limiting example, a haptic output may be used to communicate to the user the moment (e.g., precise or instantaneous moment) when contact between an electrode and a tissue of interest (e.g., nerve tissue) occurs. This can be advantageous for surgeons as they do not need to divert their gaze away from the surgical field to look at the status of the device, as they would have needed to do if status had only been communicated via visual indicators. In some arrangements, this advantage may prevent or reduce the likelihood of inadvertent injuries to nerves, such as, for example, an accidental crushing of nerve tissue from pressing too hard while diverting attention to the device.

In some embodiments, haptic output to or communication with a user may be used in conjunction with electrical impedance measurements to communicate the differing impedances of corresponding tissue (such as, for example, muscle, nerve, scar tissue, etc. In some embodiments, therefore, haptic outputs may act as an operative “extension” of a surgeon's hand or finger.

In some embodiments, haptic communication may be used to communicate if stimulus current is flowing through, towards or within the target tissue. In some arrangements the lack of haptic feedback may indicate that something is wrong or not functioning properly (e.g., that current is not “flowing” or being delivered to the electrode). In some arrangements, this may be a substitute for a visual indicator, allowing the user to focus on the surgical field. In some arrangements, such haptic output may be used in conjunction with another type of output (e.g., a visual indicator, an audible output, etc.).

In some arrangements, a bipolar probe used in nerve location devices, wherein the probe contacts are sufficiently close to one another, may be electrically shorted in the presence of interstitial fluid, saline, or other electrically conductive bodily fluids, fluids used during surgical procedures or other conductive substances. In such situations, any of the device embodiments disclosed herein can be configured to provide haptic outputs to communicate the status of an electrically shorted probe. In some arrangements, such a communication may be configured to prompt the user (e.g., simply to alert the user of the shorting issue or possible shorting issue, to instruct the user to remove the conductive fluid between the probes and/or to take any other remedial or precautionary action). As with other events or measurements disclosed herein, the user can be prompted about the electrical short (or risk thereof) with a unique output (e.g., haptic, visual, etc.).

In some embodiments, continued probing of the tissue (e.g., holding an electrode on a nerve to obtain a sustained contraction) may be indicated to the user via a specific haptic output such as, in one example, intermittent or pulsed vibrations. In some arrangements, such a unique or identifiable output to inform the user that output is still being provided (e.g., without providing a burdensome amount of haptic feedback to the user). In such examples, continued probing may include holding the probe on the tissue for a minimum or threshold value (e.g., at least 0.1, 0.2, 0.3, 0.4, 0.5, 0.6, 0.7, 0.8, 0.9, 1 seconds), as described further below.

In some embodiments, a first haptic output (e.g., based on the way a single haptic or vibratory device or component is operated, based on the particular haptic or vibratory device or component is used, etc.) is used to denote a first confirmatory event or condition (e.g., the device is turned on, the device is turned off, a short is detected along the electrode assembly, contact with nerve tissue is confirmed, etc.). Further, a second haptic output (e.g., based on the way a single haptic or vibratory device or component is operated, based on the particular haptic or vibratory deice or component is used, etc.) is used to denote a second confirmatory event or condition (e.g., the device is turned on, the device is turned off, a short is detected along the electrode assembly, contact with nerve tissue is confirmed, etc.). The first haptic output can be the same or different than the second haptic output. In some embodiments, the first haptic output is different than the second haptic output in at least one manner (e.g., a number of perceived vibrations, a vibration sharpness, a vibration duration, a vibration intensity, a vibration frequency, etc.). In some embodiments, the first confirmatory event or condition is different than the second confirmatory event or condition. Selections of Visual and Haptic Signals

In some embodiments, visual and/or haptic indicators may be synchronized to enhance a user's experience and understanding of the device function. In one non-limiting example, with reference to FIG. 1B, a traveling light pulse through a plurality of visual indicators may be synchronized with a haptic output as a train of pulses are being delivered. In some instances, this can be advantageous as it not only visually provides a user with a cue of stimulus traveling from the device to the tissue, but also provides a physical feeling through a haptic output that stimulus is being output from the device.

In another example, while a user is holding a probe to a target nerve, the device is configured to output a frequency burst 118 instead of a continuous pulse train. Such a burst may be, for example, but not limited to, a 500 ms burst of 20 Hz, as shown in FIG. 5A (e.g., with a 1.0 mA stimulation amplitude). In some embodiments, such a burst can effectively provide a tetanic contraction and can provide improved or enhanced performance. In some arrangements, the burst duration may be less than 500 ms (10, 20, 100, 200, 250, 300 ms, 10 to 100, 100 to 200, 200 to 300, 300 to 400, 400 to 500, 10 to 500, 0.1 to 200, 0.1 to 300, 0.1 to 500 ms, values and ranges between and/or within the foregoing, etc.) or greater than 500 ms, as desired or required by a particular application or use. In some embodiments, the burst duration does not exceed 2 seconds (501, 502, 505, 510, 550, 600, 700, 1000, 1500, etc.). In some instances, durations longer than 2 seconds may result in the user removing the probe from the target tissue, because the delay is perceived as too long and the user may believe no output is being delivered and resort to tapping the target tissue. With continuous frequency output, holding a probe on the nerve can induce a tetanic contraction, but at times a surgeon may not observe the response if the muscle is small (e.g., because their attention would be on the surgical field and not a distal muscle). According to some embodiments, the contraction is (or may need to be) assessed qualitatively (e.g., so the extent of muscle excursion would be important). Often times, gaze diversion can become a problem since the surgeon must observe the contraction from start to finish. Continuous frequency devices may then require the surgeon to remove the probe from the tissue and place it back on, in effect creating a tapping effect. Burst frequency becomes advantageous as the surgeon no longer has to keep lifting and tapping the probe 102 on the tissue, but may instead keep the probe in contact with the target tissue and observe the contraction take place as many times as desired. Burst frequency can be incorporated into any of the nerve locator device embodiments disclosed herein and equivalents thereof.

Furthermore, in some embodiments, with every burst output, a haptic output may be provided which is synchronized or substantially synchronized with a muscle contraction. This can be further advantageous as it may provide the user with confirmation that the electrode is in contact with the target tissue and providing stimulation to it.

In some arrangements, the combination of burst output with haptic and visual feedback may allow for the user and/or a surgical assistant or other attendant to observe a confirmatory signal that stimulation is being output.

In some embodiments, the duration of burst output, the frequency of output, the time between bursts and/or other information may be communicated through visual and/or haptic output or means.

In some embodiments, a user may be holding the probe on the target nerve to elicit a bursting response. However, the user may remove the probe from the target nerve between burst outputs. In order to communicate to the user that the probe was removed, the controller 150 of the nerve locator device 100 may be configured to sense when the probe was removed from (e.g., is no longer contacting) tissue. In one embodiment, this can be accomplished by continuously polling or otherwise querying or checking with the stimulation output to detect when current is flowing in the circuit. However, during an off period in the burst sequence, current may not be flowing, yet the user is holding the probe to or otherwise relative to the target nerve. Under such conditions, if the locator would indicate that current is not flowing, this may trigger the user to inadvertently remove the probe, thinking something is wrong. In some arrangements, the stimulation system is configured to output a low amplitude signal, herein described as a detection waveform 120, shown in, for example and without limitation, FIG. 5B, during these off periods that can be used to detect circuit continuity and provide assurance to the user that current will still flow during the burst sequence.

In some embodiments, the detection waveform 120 may be or include a square wave, sine wave, triangular wave, ramp wave, or any repetitive or non-repetitive waveform. In some arrangements, the waveform may have equal positive and negative amplitudes or have an offset such that there is no negative or positive component, or have an offset where there is a partial negative or positive component.

In some embodiments, the stimulation pulse itself may serve as a detection waveform. In this embodiment, measuring and informing the user based on the continuity of one single pulse may result in a false detection of no current flow. Thus, it may be more advantageous, according to some embodiments, to average or measure multiple pulses or count a set number of pulses (e.g., 2, 3, 4, 5, 10, 20, 50, etc.) to remove or reduce effects or impacts of noise on the signal of interest. In such embodiments, the result of pulse counting may be all or none (e.g., all measurements pass continuity or an error is displayed). In some arrangements, a threshold may be set wherein a predetermined number of pulses is or must be detected to have continuity.

In some embodiments, a moving average window may be used to determine continuity or current flow. In some arrangements, the time window of measurement may be 50 ms in duration as longer periods may result in a perceivable delay to the user. However, in other arrangements, the time window of measurement is less than or greater than 50 ms, as desired or required.

In some embodiments, the detection waveform may have an amplitude that is below the rheobase value of common nerve fibers (e.g., <50 μA, an essentially sub-threshold value so an action potential is not generated, etc.). In some arrangements, the frequency of the detection waveform may be greater than the stimulation frequency of the nerve locator device 100. In some arrangements, the stimulation frequency may be >200 Hz or greater than the firing frequency of a target nerve, such that no action potentials are generated in the nerve.

During delivery of electrical stimulation to tissue, an unwanted accumulation of charge may build up or otherwise accumulate at and/or near the electrode tissue interface. In some embodiments, charge balancing may be desirable in order to maintain a net zero charge delivery to the tissue (e.g., as excess charge may damage tissue). To accomplish charge balancing, in some embodiments, a stimulation circuit may employ active or passive charge balancing. Active charge balancing is where the amount of injected current into the tissue is measured, and a reverse polarity charge is injected to balance out the total charge to zero. Passive charge balancing typically utilizes a capacitor to block DC signals and a discharge circuit to release the built-up charge at the electrode tissue interface. In some embodiments, the discharge circuit is typically discharging in the opposite direction than the charge injection in order to balance out the net charge.

In some embodiments, where an AC waveform is used as a detection waveform 120, continuity may be sampled and measured at each positive or negative peak of the waveform. In some arrangements, the values measured at each sampling point may be counted or averaged to determine if continuity is present, e.g., similar to what was described herein. In some embodiments, arrangements where the AC waveform is symmetrical or substantially symmetrical around zero are simpler to measure than those systems where a DC offset is used. In these arrangements, balancing charge to ensure no net buildup of harmful charge takes place can be important.

In some embodiments, where charge balancing needs to be employed with a DC offset detection waveform, the charge balancing may be performed at a specified frequency (e.g., such as every period of the detection waveform, multiple periods, or a predetermined time not associated with the waveform). In some arrangements, where a burst stimulation pulse is used, as previously described, the detection waveform is only output during the off periods of the burst and charge balancing may be employed specifically during this detection waveform phase and not during the stimulation phase. According to some arrangements, during the phase where stimulation is output, the stimulation pulse itself may serve as a method to detect continuity without the need for a detection waveform.

### Example Block Diagram

Referring to FIGS. 1A and 1D, one embodiment of a block diagram of the nerve locator device of FIG. 1A is shown generally at 100 in FIG. 1D. In this embodiment, the controller 150 is in communication (e.g., electrical, data and/or operative communication) with user input controls shown generally at 130, a power supply system shown generally at 140, an electrode power system shown generally at 160, the visual signal generator 170 and a non-visual signal generator, which in this embodiment includes the haptic signal generator 180.

In this embodiment, the controller 150 includes a microcontroller 343, which includes an internal computer-readable medium 190 that acts as a program memory for the microcontroller, and which further includes an internal digital-to-analog converter (not shown). More generally, in this specification, including the claims, the term “controller” is intended to broadly encompass any type of device or combination of devices which the present specification and common general knowledge would enable the notional person of ordinary skill in the art to substitute for the microcontroller 343 to perform the functions described herein. Such devices may include, by way of example and without limitation, other types of microcontrollers, microprocessors, other integrated circuits, other types of circuits or combinations of circuits, logic gates or gate arrays, Programmable Logic Controllers (PLCs) or other programmable devices of any sort, for example, either alone or in combination with other such devices located within the device 100 or externally and remote from the device 100, for example.

Similarly, although the computer-readable medium 190 that acts as the program memory for the controller 150 is shown as a built-in component of the controller 150 in the embodiment illustrated in FIG. 1D, the computer-readable medium 190 may be external to the microcontroller 343 or other device that executes the functions of the controller 150. The computer-readable medium 190 may include any suitable type of computer-readable storage medium, including without limitation, flash or other solid-state memory, programmable read-only memory, or ferroelectric or other non-volatile random access memory, for example.

In such embodiments, the computer-readable medium 190 stores one or more routines, the routines including computer-readable instructions, which, when executed by the controller 150, can cause the controller 150 to control and/or otherwise communicate with any one or more of the input controls 130, the power supply system 140, the electrode system 160, the visual signal generator 170, the haptic signal generator 180, etc. (e.g., to cause each of the various functions of the device 100 described herein to be carried out or otherwise executed). In some embodiments, the instructions stored in the computer-readable medium 190 thereby configure or program the controller 150 to cause the various functions of the device 100 described herein to be carried out. Accordingly, throughout this description, wherever the device 100 is described as performing a particular function, whether automatically or in response to user input received via the input controls 130, it is to be understood that the controller 150, as configured by the instructions stored in the computer-readable medium 190, and in conjunction with whichever one or more of the electrode system 160, the visual signal generator 170 and/or the haptic signal generator 180 are associated with that particular function, collectively act as means for performing that particular function.

In one embodiment, the user input controls 130 shown in FIG. 1D include the user input selector 106 and the switch 114 or other control, as shown in FIG. 1A.

In some embodiments, the power supply system 140 includes a battery 340 and a power management system 341. More particularly, in certain embodiments, the battery 340 includes one or more batteries (e.g., a single AAAA battery). In alternative embodiments, the battery 340 may include one or more individual batteries, and the battery or batteries may include sizes, voltages and physical form factors different than those of the illustrative example that has a single AAAA battery, as desired or required.

In some embodiments, the electrode system 160 includes a constant current source 345 that is configured to supply a constant electrical current to the electrodes 122 of the electrode system 160 during stimulation of the tissue, as directed by the controller 150 as described herein. In this regard, the constant current control of the electrical stimulation can be advantageous, because, for instance and without limitation, it tends to maintain steady or substantially steady activation of a nerve irrespective of environmental conditions around and/or relating to the nerve (e.g., a change in electrical impedance due to fluid inflow). Alternatively, in other embodiments, constant current control can be omitted or avoided. For example, some such embodiments may substitute constant voltage control.

In the present embodiment, the visual signal generator 170 of FIG. 1D includes the visual indicators (LEDs) 108 and the display window 112, as shown in FIG. 1A, for example.

In such embodiments, the non-visual signal generator shown in FIG. 1D can include the haptic signal generator 180 of FIG. 1A. More particularly, in this embodiment, the haptic signal generator 180 can include only a single vibratory motor, such as a linear resonant actuator, an eccentric rotating mass, a linear magnetic ram, or a piezo vibration actuator, for example. Alternatively, other embodiments may employ one or a plurality of vibratory motors of the same or different types at different locations within the device 100.

### EXAMPLES

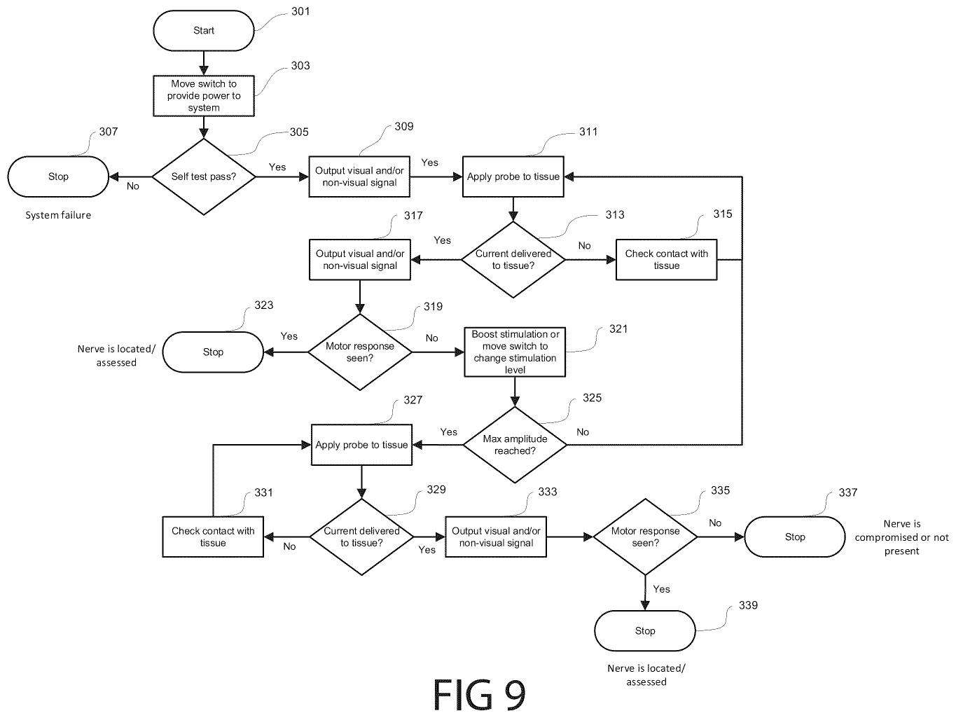

A non-limiting example of nerve locator device usage embodying several of the described concepts is outlined herein and shown in a flow diagram in FIG. 9. FIG. 9 illustrates a combination of some steps performed by a user such as a surgeon and some complementary steps performed by the device 100 in response to the user's inputs. In some arrangements, at steps 301 and 303, the surgeon may initially power on the device 100 using a multi-pole switch 114 located towards the proximal end of the device. In some embodiments, the switch or other control of the device is moved a single position to enable the initial output setting. In response, the device 100 may cause the controller 150 to execute a self-test at step 305, which if failed, in one embodiment, will stop the operation of the device 100 at step 307, and which if passed will cause the device 100 to provide a haptic vibration and illuminate a visual indicator to communicate a power-on status at step 309.

With continued reference to the embodiment illustrated in FIG. 9, at step 311, the surgeon may then probe the tissue of interest while observing for a muscular contraction distal or near the probing area. Haptic vibration, either alone or in conjunction with a visual indicator, can be used to provide feedback to the surgeon that current is flowing from anode to cathode. At step 313, in one embodiment, the controller 150 of the device is configured to determine whether electrical current is flowing through the electrode system 160. In this embodiment, if no current is flowing then there is no haptic feedback or visual illumination, and at step 315, the surgeon verifies whether the electrodes 122 of the electrode system 160 are in contact with tissue, and the surgeon returns to step 311 to apply the probe 102 to the tissue. In some embodiments, if at step 319 the surgeon observes the desired motor response, then the surgeon concludes that the nerve has been located and assessed, and may then stop using the device 100. According to one embodiment, if at step 319 the surgeon fails to observe any motor response, for example, if the output power is insufficient to cause any muscular response or if a tissue that is suspected to be a nerve is not resulting in a muscular response when probed, then the surgeon may increase the output power at step 321 (e.g., in one of two ways).

According to a first method embodiment, an actuator is located at the fingertips of the surgeon (e.g., such as the user input selector 106) that may function to double the output power. Such a relatively quick interaction with the actuator may serve as a rapid comparison when probing tissue with and without the actuator activated, essentially providing two tests at the surgeon's fingertips while maintaining contact with tissue. In some embodiments, during activation of the actuator, haptic vibration will differ from when the actuator is not activated. An increased vibration may be used to communicate increased power output. In some embodiments, this may be accomplished through a faster vibration, a more forceful vibration, a different length of vibration and/or the like, as desired or required.

According to some embodiments, in addition to haptic vibration, one or more visual indicators may also be used in a nerve location device, either in addition to or in lieu of a haptic indictor or output. Such a visual indicator can be configured to change color, pulsing frequency and/or another output effect to indicate greater amount of output power. In some embodiments, at step 325, if the output power is still insufficient but the maximum (or other upper threshold) power setting has not yet been selected, the surgeon may move the switch 114 or other control (e.g., located on the proximal aspect 101 of the device) to the next setting (e.g., discrete or non-discreet setting). Such a change in setting increases output power, according to one embodiment.

In some embodiments, the surgeon may then repeat probing of tissue at step 327 and contact checking at step 331, and the device 100 may repeat current checking at step 329 and the generation of visual and haptic signals at step 333 in an effort to detect the desired motor response at step 335, in the same general manner as described herein in connection with steps 311, 313, 315, 317 and 319. In some embodiments, if the desired motor response is observed at step 335, then it is concluded at step 339 that the nerve has been located and assessed and the user may stop operating the device 100. In certain arrangements, if the desired motor response is not observed at step 335, despite maximum power having been reached at step 325, then it is concluded at step 337 that either the nerve is compromised or is not present.

According to some embodiments, when a surgeon locates a target nerve of interest, it may be desirable to observe the functionality of the muscle that is connected to the target nerve. This may be performed by, for example, tapping the probe on and off the nerve in order to create a brief tetanic contraction resulting in the muscle contracting and relaxing when the probe is removed from the tissue. However, as discussed above, the physical act of tapping may require the surgeon's area of high visual acuity to be focused on the target nerve. Advantageously, in certain embodiments, the need for tapping can be avoided or eliminated. Instead, in some arrangements, the embodiments disclosed herein and variations thereof can permit the surgeon to hold the device in place and have the locator automatically output a tetanic burst waveform such that the nerve probe does not need to be placed on and off the nerve repeatedly. This can also allow the surgeon to focus on the muscle and observe the functional response. In some embodiments, the burst output may result in a differentiable vibratory output to signal to the user that a burst output waveform is being output. In addition to haptic output, the device can include a visual indicator. In some arrangements, such a visual indicator can be configured to illuminate in a different color, pulsing frequency and/or another output property or effect. In such embodiments, when the probe is removed from the tissue, a differentiable haptic vibration can be communicated to the user to inform them that the probe was removed and burst output is inactive. In one embodiment, this haptic vibration will be at an amplitude, frequency, or duration that is noticeably different (e.g., lower, less sharp, double, sharper, higher, etc.) than that when the probe is applied to the tissue. This can be advantageous because a surgeon may be holding the probe on the tissue for a duration that is considerably longer than a simple tap, and while diverting their gaze to the muscle, may not realize the probe was removed inadvertently from the tissue. In some embodiments, while a lack of vibration can provide one cue that the probe was removed, a differentiable vibration for removal during burst stimulation may be preferable to distinguish removal of the probe from tissue during standard tapping.

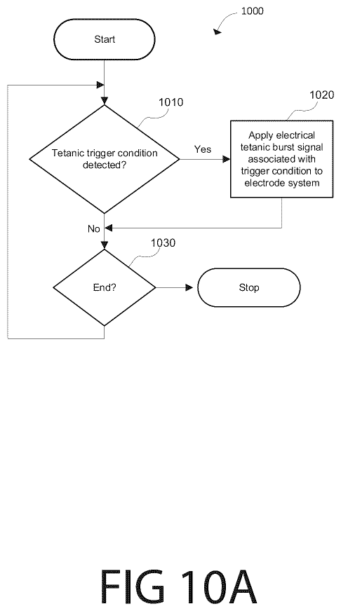

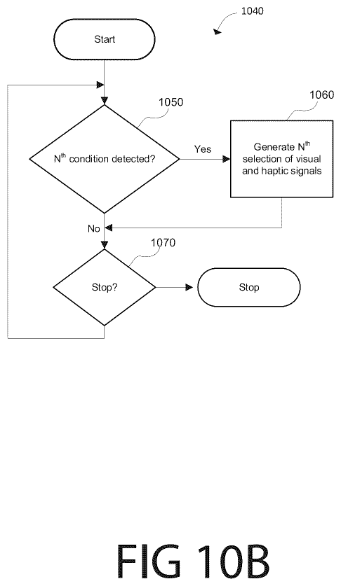

Referring to FIGS. 1A, 1D and 10A, an illustrative example of a routine for controlling the device 100 to produce such an electrical tetanic pulse stimulation to tissue, is shown generally at 1000. In this embodiment, the routine 1000 includes computer-readable instruction codes stored in the computer-readable medium 190 of the controller 150, which for the purpose of the present embodiment can be simplified into a plurality of blocks of instructions codes, which when executed by the controller, cause a plurality of different corresponding functions to be carried out. In an illustrative embodiment, the routine 1000 begins with a first block of codes 1010, which directs the controller 150 to detect a trigger condition associated with a predefined tetanic burst stimulation, and automatically apply an electrical tetanic burst signal associated with the predefined tetanic burst stimulation to the electrode system of the hand-held device, in response to the detecting of the trigger condition. For example, block 1010 may configure and direct the controller to detect the trigger condition by detecting continuous contact between the electrode system and the tissue for at least a predefined duration of time. Alternatively or supplementarily, the trigger condition may include one or more predefined conditions for input signals from the input controls 130, such as the first user input selector 106 and the slide switch 114, for example.

In some embodiments, if at block 1010 a tetanic trigger condition is detected, a second block 1020 of codes directs the controller 150 to automatically apply an electrical tetanic burst signal associated with the predefined tetanic burst stimulation with which the detected trigger condition is associated, to the electrode system 160 of the hand-held device 100, in response to the detecting of the trigger condition. For example, in an illustrative embodiment, block 1020 configures and directs the controller 150 to apply (e.g., automatically apply) the electrical tetanic burst signal by generating an electrical signal burst having a duration between about ten milliseconds and two seconds. More particularly, in some embodiments, the duration is about one half second (500 ms). However, in other embodiments, the duration can be less or more than 500 ms.

According to some embodiments, block 1030 may direct the controller 150 to help determine whether a pre-defined user input or user action (e.g., removal of the probe from tissue), indicating, for example, a desire to exit from the tetanic burst mode has been received by the controller 150 from the user input controls 130, and if so to exit accordingly, and otherwise to continue processing at block 1010 to detect a new tetanic trigger condition.

Referring to FIGS. 1A, 1D and 10B, in an illustrative embodiment, an example of a method or routine for operating the device 100 is shown generally at 1040. In this embodiment, the routine 1040 includes computer-readable instruction codes stored in the computer-readable medium 190 of the controller 150, which for the purpose of the present embodiment can be simplified and grouped into a plurality of blocks of instructions codes, which when executed by the controller 150, cause a plurality of different corresponding functions to be carried out. In an illustrative embodiment, the routine 1040 begins with a first block of codes 1050, which configures or directs the controller 150 to detect a first condition pertaining to the device 100 (such as a device status condition or alarm condition, for example) or to the stimulation of the tissue by the device 100. More particularly, in this embodiment block 1050 directs the controller 150 to detect any one (or an “nth” one) of a plurality of different predefined conditions pertaining to the device 100 or to the stimulation of the tissue.

According to some embodiments, the routine 1040 defines associations, by which each one of the predefined conditions is associated with a specific and potentially different respective corresponding selection of visual and haptic signals to be generated by the visual signal generator 170 and the haptic signal generator 180 under the direction of the controller 150, in response to detecting that specific predefined condition. In this embodiment, the predefined conditions detected at block 1050 include one or more conditions selected from the group consisting of: electrical current flow between the electrodes 122 of the electrode system 160; a change to any one of the amplitude, frequency or duration of the electrical stimulation being provided by the electrode system 160; battery depletion of the battery 340; battery error or other failure of the power supply system 140 (e.g., system voltage error); electrode error associated with the electrodes 122 of the electrode system 160; electrode shorting of the electrodes 122; and a system status change to any one of power status, standby status, wake status and wake time for the device 100.

In such embodiments, the predefined conditions detected at block 1050 further include one or more conditions selected from the group consisting of: a detection of a nerve in the tissue, a commencement of contact between the electrodes 122 and the tissue; a pulse of the electrical stimulation of the tissue; a frequency burst of the electrical stimulation of the tissue; a change in electrical impedance; and a duration of the stimulation reaching a predefined threshold. In this embodiment, each of the predefined conditions detectable at block 1050 is associated by the routine 1040 with a respective corresponding selection of visual and haptic signals from among those that the visual and haptic signal generators 170 and 180 are capable of generating. According to some arrangements, each condition is typically associated with a different selection of visual and haptic signals, though not necessarily uniquely. For example, in some embodiments a particular selection of visual and haptic signals may be associated with a group or category of predefined conditions, if desired.

With further reference to this embodiment, in response to detecting one of the predefined conditions associated with the device 100 or with the stimulation of the tissue by the device 100 at block 1050, block 1060 directs the controller 150 to determine the specific selection of visual and haptic signals that is associated with the detected condition by the routine 1040. In one embodiment, block 1060 then directs the controller to control the visual signal generator 170 and the haptic signal generator 180, to thereby generate with the hand-held device, the determined selection of visual and haptic signals associated with the detected condition.

In some embodiments, unless a stop input is detected by the controller 150 at block 1070, processing continues iteratively back to block 1050, which directs the controller 150 to continue detecting any of the predefined conditions pertaining to the device 100 or to the stimulation of the tissue by the device. According to some arrangements, after a first execution of blocks 1050 and 1060 to detect a first condition and generate a first corresponding selection of visual and haptic signals associated with the first condition, a subsequent successful (“yes”) execution of block 1050 configures or directs the controller 150 to detect a second condition pertaining to the device 100 or to the stimulation of the tissue. In some arrangements, in response to detecting the second condition at block 1050, block 1060 directs the controller 150 to control the visual and haptic signal generators 170 and 180 to generate with the hand-held device 100, a second selection of visual and haptic signals associated with the second condition. In this example, the first condition is different than the second condition, and the first selection of visual and haptic signals is different than the second selection of visual and haptic signals.

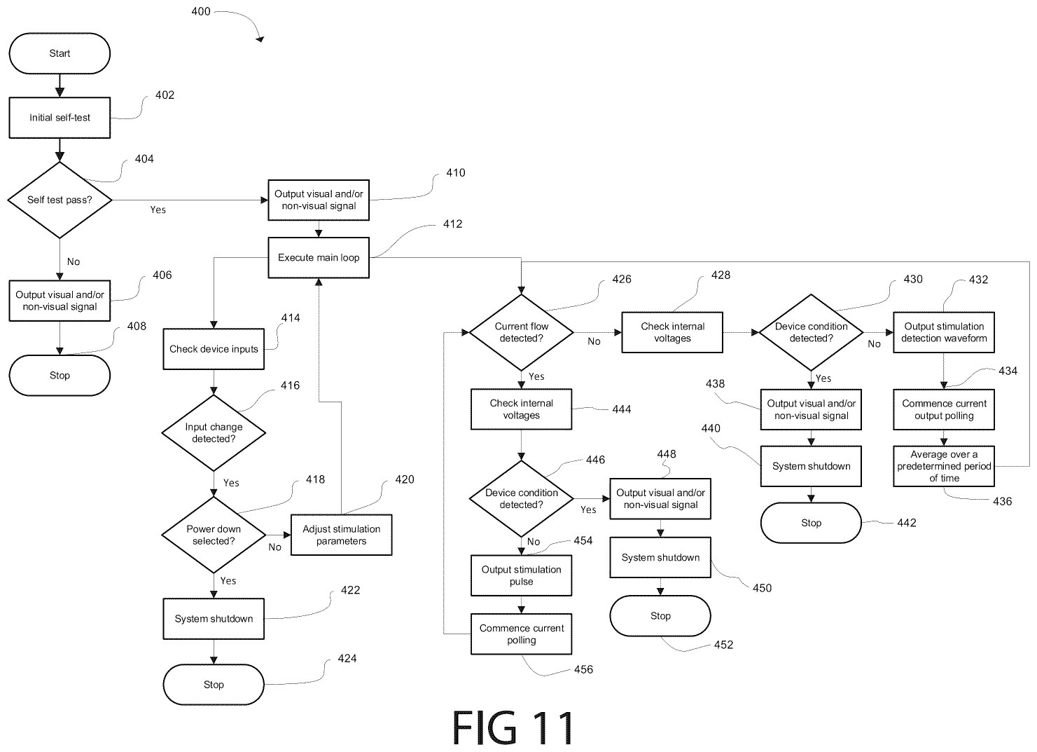

Referring to FIGS. 1A, 1D and 11, an illustrative example of a main operation routine for controlling the operation of the device 100 is shown generally at 400. In this embodiment, the main operation routine 400 includes blocks or groups of instruction codes stored in the computer-readable medium 190, which when executed by the controller 150, causes the device to perform the various functions described herein. In one embodiment, the system is initially powered on and a first block 402 of instruction codes directs the controller 150 to perform a power-on self-test. Block 404 directs the controller 150 to determine whether the self-test has resulted in an error and if not, the device is considered to pass the self-test, in which case block 410 directs the controller 150 to control the visual and haptic signal generators 170 and 180 to generate a selection of visual and/or non-visual signals 410 associated with the event (detected condition) of passing the self-test. In some embodiments, if the self-test fails at block 404, block 406 directs the controller 150 to control the visual and haptic signal generators 170 and 180 to output a different visual and non-visual signal, and block 408 then directs the controller 150 to turn off the device 100.

According to some embodiments, following a pass in the self-test at blocks 404 and 410, block 412 directs the controller 150 to execute a main loop including two concurrent subroutines to enable the features described above. In some arrangements, in a first subroutine, blocks 414 and 416 direct the controller 150 of the device 100 to continually check for changes to the input controls 130 and if a change is detected, block 418 directs the controller 150 to determine whether one of the input controls 130, such as the slide switch 114, has been changed by a user of the device to a power-down position. In one embodiment, if the power-down was not selected then block 420 assumes that the change in the input controls 130 represents a request to adjust a stimulation parameter, and block 420 therefore directs the controller 150 to control the electrode system 160 to adjust a currently selected stimulation parameter, as described elsewhere herein. In this regard, according to some arrangements, the user input controls 130 including user input selector 106 and switch 114 for example, may be actuated by a user to select and then adjust different stimulation parameters. For example, depending on the current settings of the user input controls 130, upon detecting the change in input controls at blocks 416 and 418, block 420 may direct the controller 150 to adjust at least one of an amplitude, a pulse width, a duration, an output energy, a frequency, and a burst frequency, of the electrical stimulation, in response to the changed detected in the user input controls.

According to some embodiments, if a power-down selection is detected at block 418, then blocks 422 and 424 direct the controller 150 to commence a system shutdown of the device 100. In some arrangements, concurrently, in a second concurrent subroutine, a first block 426 of codes directs the controller 150 to determine if current is flowing between the electrodes 122 of the electrode system 160 (or between an electrode and a return contact for mono-electrode embodiments). In some embodiments, if no current flow is detected at block 426, then block 428 directs the controller 150 to check the internal voltages such as the system voltage, battery voltage, high voltage, and any other voltages present, and block 430 directs the controller 150 to check for error conditions or other abnormal responses. According to some embodiments, if an abnormal response is detected, block 438 directs the controller 150 to control the visual and haptic signal generators 170 and 180 to output an error visual and non-visual signal and blocks 440 and 442 direct the controller 150 to shut down the device 100. On the other hand, in some embodiments, if at blocks 428 and 430 the voltages are in the correct range, block 432 directs the controller 150 to control the electrode system 160 to output a stimulation detection waveform, and block 434 directs the controller 150 to commence continuous current output polling of the current flowing through the electrode system 160. In some embodiments, block 436 directs the controller 150 to average the responses of the output polling over a pre-determined time to remove any noise from the response. In certain arrangements, processing returns back to block 426 where current flow is then polled again, and if found to be true then blocks 444 and 446 direct the controller 150 to check internal voltages again, in the same manner as described above in connection with blocks 428 and 430.

According to some embodiments, if at block 444 the voltages are in the correct range and no anomalous device conditions have been detected at block 446, then block 454 directs the controller 150 to control the electrode system 160 to generate a stimulation pulse 454, and block 456 directs the controller to commence current polling similar to block 434 above. In some embodiments, processing then returns to block 426. On the other hand, in one arrangement, if at blocks 444 and 446 an abnormal response is detected, block 448 directs the controller 150 to control the visual and haptic signal generators 170 and 180 to output an error visual and non-visual signal and blocks 450 and 452 direct the controller 150 to shut down the device 100.

Multiple Haptic and/or Visual Signals

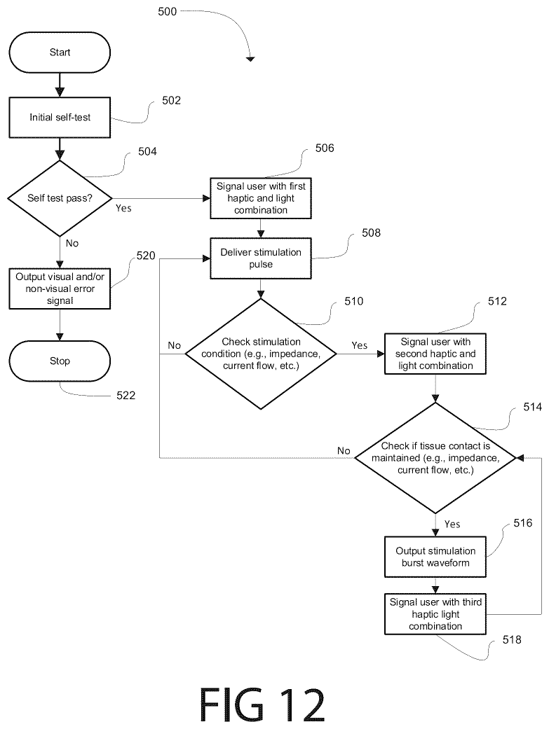

Referring to FIGS. 1A, 1D and 12, a tetanic stimulation routine according to an illustrative embodiment of the present disclosure is shown generally at 500 in FIG. 12. In this embodiment, the tetanic stimulation routine 500 includes blocks or groups of instruction codes stored in the computer-readable medium 190, which when executed by the controller 150, cause the device to perform the various functions described herein. According to some embodiments, the system is initially powered on, and blocks 502 and 504 direct the controller 150 to execute a power-on self-test and determine whether the device 100 has passed the self-test. In one arrangement, if no errors are detected at blocks 502 and 504 then the device 100 is considered to pass the self-test, and block 506 directs the controller 150 to control the visual and haptic signal generators 170 and 180 to generate a first selection of haptic and light/visual signals associated with the condition or event of passing the self-test. According to some arrangements, if the self-test fails at block 504, then block 520 directs the controller 150 to control the visual and haptic signal generators 170 and 180 to generate a different visual and non-visual signal, more particularly a selection of visual and haptic signals which is associated with the failure of the self-test and which is different than the selection of visual and haptic signals discussed above in connection with block 506, and block 522 directs the controller 150 to turn off the device 100.

According to some embodiments, following a pass in the self-test at block 504 and corresponding visual-haptic signaling of the pass at block 506, block 508 directs the controller 150 to control the electrode system 160 to output a stimulation pulse, and block 510 directs the controller 150 to check a stimulation condition such as current flow, impedance, etc. In some arrangements, if the stimulation condition is detected or passes at block 510, then block 512 directs the controller 150 to control the visual and haptic signal generators 170 and 180 to generate a further selection of visual and haptic signals associated with the detection of the stimulation condition. In one embodiment, the selection of visual and haptic signals at block 512 is different than those generated by the device 100 at blocks 506 and 520 above. In some embodiments, an illustrative example of a stimulation condition is detecting that the electrodes of the electrode system 160 have maintained contact with the tissue for a predefined amount of time. Following detection of the stimulation condition at block 510 and related signaling at block 512, in certain arrangements, block 516 directs the controller 150 to control the electrode system 160 to generate a burst waveform as previously described, and block 518 directs the controller 150 to control the visual and haptic signal generators to generate a third selection of visual and haptic signals associated with the burst waveform. In some embodiments, if the tissue contact check at block 514 fails or the stimulation condition is not detected at block 510, the device 100 will continue to output a regular stimulus pulse as described in connection with block 508.

Although several embodiments and examples are disclosed herein, the present application extends beyond the specifically disclosed embodiments to other alternative embodiments and/or uses of the various inventions and modifications, and/or equivalents thereof. It is also contemplated that various combinations or subcombinations of the specific features and aspects of the embodiments may be made and still fall within the scope of the inventions. Accordingly, various features and aspects of the disclosed embodiments can be combined with or substituted for one another in order to form varying modes of the disclosed inventions. Thus, the scope of the various inventions disclosed herein should not be limited by any particular embodiments described above. While the embodiments disclosed herein are susceptible to various modifications, and alternative forms, specific examples thereof have been shown in the drawings and are described in detail herein. However, the inventions of the present application are not limited to the particular forms or methods disclosed, but, to the contrary, cover all modifications, equivalents, and alternatives falling within the spirit and scope of the various embodiments described and the appended claims. Further, the disclosure herein of any particular feature, aspect, method, property, characteristic, quality, attribute, element and/or the like in connection with an implementation or embodiment can be used in all other implementations or embodiments set forth herein.

In any methods disclosed herein, the acts or operations can be performed in any suitable sequence and are not necessarily limited to any particular disclosed sequence and not be performed in the order recited. Various operations can be described as multiple discrete operations in turn, in a manner that can be helpful in understanding certain embodiments; however, the order of description should not be construed to imply that these operations are order dependent. Additionally, any structures described herein can be embodied as integrated components or as separate components. For purposes of comparing various embodiments, certain aspects and advantages of these embodiments are described. Not necessarily all such aspects or advantages are achieved by any particular embodiment. Thus, for example, embodiments can be carried out in a manner that achieves or optimizes one advantage or group of advantages without necessarily achieving other advantages or groups of advantages.

The methods disclosed herein include certain actions taken by a practitioner; however, they can also include any third-party instruction of those actions, either expressly or by implication. For example, actions such as “locating” a nerve, “coupling” an electrode to a nerve, “initiating” a stimulation procedure include “instructing locating,” “instructing coupling,” and “instructing initiating” etc., respectively. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1 mm” includes “1 mm.” Phrases preceded by a term such as “substantially” include the recited phrase and should be interpreted based on the circumstances (e.g., as much as reasonably possible under the circumstances). For example, “substantially rigid” includes “rigid,” and “substantially parallel” includes “parallel.”

1. A method of operating a device configured to apply electrical stimulation to tissue, the method comprising:
generating a first haptic output signal configured to be detected by a user holding the device, the first haptic output signal being associated with a first condition pertaining to the device or to the electrical stimulation, in response to detecting the first condition; and
generating a second haptic output signal configured to be detected by a user holding the device, the second haptic output signal being associated with a second condition pertaining to the device or to the electrical stimulation, in response to detecting the second condition;
wherein the first condition is different than the second condition; and
wherein the first haptic output signal is different than the second haptic signal.
